(19) United States
(12) Patent Application Publication
(10) Pub. No.: US 2025/0207524 A1
(43) Pub. Date: Jun. 26, 2025

(54) Turbine NOx Sensor

(71) Applicants: Cummins Ltd, London (GB); Cummins Emission Solutions, Inc., Columbus, IN (US)

(72) Inventors: Chetan Kishorrao Chawane, Pune (IN); Enoch Nanduru, Pune (IN); Sagar Shekhar Andhari, Pune (IN); Donald Edward Willey, West Yorkshire (GB); Paul T. Frisch, Columbus, IN (US); Jim L. Alonzo, Columbus, IN (US); Matthew Robert Brookes, West Yorkshire (GB)

(21) Appl. No.: 18/848,104

(22) PCT Filed: Mar. 16, 2023
(86) PCT No.: PCT/GB2023/050637

§ 371 Date: Sep. 17, 2024

(30) Foreign Application Priority Data
Mar. 17, 2022 (GB) 2203750.1

(51) Int. Cl. F01N 13/00; F02B 77/08

(52) U.S. Cl. CPC F01N 13/008; F02B 77/086; F01N 2560/026

(57) ABSTRACT

There is a provided an exhaust gas conduit for an exhaust system of an internal combustion engine. The exhaust gas conduit includes a main passage for a main flow of exhaust gases passing through the exhaust gas conduit, a chamber configured to receive an aliquot of exhaust gases separated from the main flow of exhaust gases. A mounting point is provided in the chamber for mounting an exhaust gas sensor. The chamber being configured to modify the velocity and/or the pressure of exhaust gases passing therethrough.

The present invention relates to an exhaust gas conduit for an exhaust system of an internal combustion engine, a turbine housing comprising such an exhaust gas conduit, a turbocharger comprising such an exhaust gas conduit or turbine housing, an internal combustion comprising such a turbocharger, turbine housing or exhaust gas conduit, a method of measuring a property of an exhaust gas of an internal combustion engine, an alternative exhaust gas conduit for an exhaust system of an internal combustion engine and a shield for an exhaust gas sensor. The present invention has particular, but not exclusive, application to turbocharged internal combustion engines.

Turbochargers are used within internal combustion engine systems to increase the pressure of the intake air entering the internal combustion engine to a pressure above atmospheric pressure. This is known as a “boost pressure”. By increasing the pressure of the intake air entering the internal combustion engine, more oxygen is available within the internal combustion engine to support the combustion of a larger amount of fuel, and therefore increases the amount of power produced by the engine.

Turbochargers comprise a compressor and a turbine. The compressor comprises a compressor wheel configured to impart energy to an incident fluid stream, and the turbine comprises a turbine wheel configured to extract energy from an incident fluid stream. The compressor wheel and the turbine wheel are attached to opposite ends of a turbocharger shaft, such that the two rotate in unison. The compressor receives intake air from the atmosphere and delivers the intake air to an intake manifold of the internal combustion engine. The turbine receives exhaust gas from an exhaust manifold of the internal combustion engine and delivers the exhaust gas to an aftertreatment system. During use, exhaust gas leaving the internal combustion engine passes through the turbine, causing the turbine wheel to rotate. The rotation of the turbine wheel drives the compressor wheel, which acts to compress the intake air as it is delivered to the intake manifold.

Exhaust gases from internal combustion engines contain substances that are harmful to the environment. Most countries have vehicle emission standards which limit the amount of such substances that an internal combustion engine system is permitted to emit. Consequently, modern internal combustion engine systems comprise exhaust gas aftertreatment systems designed to remove harmful substances from the exhaust gas.

Typically, an exhaust gas aftertreatment system will comprise a particulate filter and one or more catalytic reducers. The particulate filter removes heavy combustion products, e.g. soot, from the exhaust gas. The catalytic reducers remove harmful substances such as Nitrogen Oxides (NOx) from the exhaust gas. Catalytic reducers generally comprise a large number of narrow channels made from a material selected to support a chemical reaction that removes NOx from the exhaust gas. The narrow channels provide a large surface area for the catalytic reaction to take place. Several kinds of catalytic reducers are available on the market, such as two-way catalytic reducers, three-way catalytic reducers, diesel oxidation catalytic reducers (DOCs), and selective catalytic reducers (SCRs). DOCs and SCRs are typically employed in diesel engine systems. For the SCRs specifically, in order for the SCR reaction to work, it is necessary to mix an exhaust gas aftertreatment fluid with the exhaust gas before it enters the catalytic reducer. The exhaust gas aftertreatment fluid is usually a mixture of around 30% to 35% by volume urea (CO(NH₂)₂) to about 65% to 70% by volume deionised water (H₂O). The exhaust gas aftertreatment fluid is often referred to as Diesel Exhaust Fluid (DEF) and is commonly available under the registered trade mark AdBlue.

Conventionally, the DEF is mixed with the exhaust gas in a decomposition chamber.

The DEF is injected into the decomposition chamber using a dosing module. In the decomposition chamber, heat is exchanged from the exhaust gas to the DEF which causes the water within the DEF to evaporate and the urea to thermally decompose into the reductants ammonia (NH₃) and Isocyanic Acid (HNCO) which are required to support the SCR reaction.

A typical decomposition chamber comprises a relatively large cross-sectional area in comparison to the width of standard exhaust gas ducting. Exhaust gas entering the decomposition chamber expands, causing the velocity of the exhaust gas to reduce and the pressure of the exhaust gas to increase. This rapid expansion of the exhaust gas causes the formation of turbulent vortices. DEF is then injected into the decomposition chamber, whereupon the turbulent vortices encourage mixing of the DEF with the exhaust gas. The heat exchange between the exhaust gas and the DEF causes the urea in the DEF to decompose into the reductants, and the mixture of reductants and exhaust gas is then passed to the SCR.

If the exhaust gas and DEF are not mixed well enough, the heat exchange between the DEF and the exhaust gas will not be sufficient to decompose the DEF into the required reductants. Furthermore, poor mixing means that the reductants are not evenly distributed within the flow, and therefore some channels of the catalytic reducer will not receive enough reductant to support the SCR reaction. To ensure adequate mixing, it is common for the decomposition chamber to comprise a mixing plate configured to generate additional turbulence. However, the additional turbulence caused by the mixing plate and the fluidic friction exerted by the mixing plate on the exhaust gas creates a back-pressure on the exhaust gas in the decomposition chamber. This back pressure is passed upstream and acts to increase the pumping work required by the internal combustion engine, and accordingly reduces the overall efficiency of the engine system. Accordingly there is need for improvement in this technical area.

In order to effectively convert nitrogen oxides of the exhaust gas, it is desirable to know how much DEF is required to be provided. One or more exhaust gas sensors capable of measuring the NOx concentration may be provided to measure the NOx concentration of exhaust gases, and this value can be used to determine the amount of DEF required or to otherwise provide information which may be used to control one or more parameters of an engine to control the NOx concentration in the exhaust gases. Normally, NOx sensors are located upstream of SCR units to measure the concentration of NOx gases in the exhaust to control the amount of DEF added. Additionally or alternatively, NOx sensors can be located downstream of the SCR catalyst to monitor the performance of the SCR catalyst. Exhaust gas sensors are liable to failure if they are exposed to conditions too far outside of their normal operating specifications and NOx sensors are vulnerable to effects from variations and fluctuations in exhaust gas temperature, exhaust gas pressure and exhaust gas velocity. Variations and fluctuations in the temperature, velocity or pressure of the exhaust gas may cause failure of electrical components and circuitry in a NOx sensor.

The failure of an exhaust gas sensor can lead to less efficient management of exhaust gas composition, decreased engine and exhaust system performance, as well as additional costs for the user associated with replacing the failed sensor and associated vehicle downtime.

US20210255277 describes a method for determining whether a NOx sensor in an exhaust stream is performing properly and describes how it can be determined if a NOx sensor has failed. U.S. Ser. No. 10/221,746 also describes a failure apparatus for exhaust gas control apparatus in cases where it is difficult to determine which unit within the exhaust system has failed. Even though these methods may allow the failure of an exhaust component to be determined, these methods are after the event and the sensors would necessarily already have begun to fail or failed, meaning that remedial action is required.

There exists a need to provide alternative systems which overcome one or more of the disadvantages of known systems, whether mentioned in this document or otherwise.

### SUMMARY OF THE INVENTION

According to a first aspect of the present invention, there is provided an exhaust gas conduit for an exhaust system of an internal combustion engine, said exhaust gas conduit including a main passage for a main flow of exhaust gases passing through the exhaust gas conduit, a chamber configured to receive an aliquot of exhaust gases separated from the main flow of exhaust gases, a mounting point in the chamber for mounting an exhaust gas sensor, the chamber being configured to modify the velocity and/or the pressure of exhaust gases passing therethrough.

Exhaust gas sensors are susceptible to damage caused by exposure to exhaust gases at high temperatures, pressures or velocities, particularly where these vary in a pulsatile manner. Exhaust gas sensors may be particularly susceptible to damage when exposed to exhaust gas with a high velocity. Exposure to high velocity exhaust gas can decrease the accuracy of the exhaust gas sensor and reduce the lifespan of the exhaust gas sensor. Exhaust gas sensors may comprise a ceramic element, exposing the ceramic element to exhaust gasses with pressure pulsations and/or thermal gradients may cause the ceramic element to fail. As such, in order to protect exhaust gas sensors from damage and to provide them with a longer operating lifespan, they may be provided far downstream of the exhaust manifold where the flow variability and velocity of the exhaust gases are reduced. Positioning exhaust gas sensors downstream reduces exposure of the sensors to corrosive substances, for example, Diesel Exhaust Fluid (DEF) and decomposition products, e.g. ammonia. In addition, exhaust gas sensors are positioned downstream of catalyst units which additionally reduces the magnitude of the pressure pulses in the exhaust gases created by the firing of the cylinders in the internal combustion engine. Conventionally, exhaust gas sensors are disposed to sample the main flow of exhaust gases within an exhaust system and so are exposed to the temperatures, gas velocities, and pressures of the main exhaust flow. This can lead to damage to the exhaust gas sensors and a reduction in operational lifespan.

The exhaust gas conduit according to the present invention provides for an aliquot of exhaust gases to be separated from the main flow of exhaust gases and passed into a chamber which is separate from the main exhaust passage. The term “aliquot of exhaust gases” encompasses a portion of exhaust gas separated from the main flow, including, but not limited to, a continuous stream of exhaust gas that is separated from the main flow. The term “aliquot of exhaust gases” also encompasses a minor flow or an auxiliary flow of exhaust gas that is separated from the main flow. The chamber is configured to reduce one or both of the velocity and the pressure of the exhaust gases therein, which allows an exhaust gas sensor to be positioned within the chamber and provides a less hostile environment for the sensor. This also allows the exhaust gas sensor to be positioned further upstream than would otherwise be the case. Indeed, the present invention allows for the placement of an exhaust gas sensor downstream of an outlet of a turbocharger without there being an intervening catalyst unit or filter unit between the turbocharger outlet and the exhaust gas sensor. In addition, exhaust gas sensors, particularly NOx sensors, may have their readings adversely affected by the presence of reductants added to the exhaust gases to reduce NOx levels. As such, by providing a chamber which is separate to the main flow of exhaust gases, an exhaust gas sensor disposed within the chamber is protected from the reductants added to the exhaust gases. In this way, the exhaust gas sensor is able to measure the composition of the exhaust gases without the reading being affected by the presence of reductants which have been injected into the exhaust gases. Without the protection afforded by the chamber, exhaust gas sensors, particularly NOx sensors, can be damaged by exposure to DEF, can provide incorrect readings, and in the worst case can fail, thereby requiring replacement of said sensor.

The chamber being configured to modify the velocity and/or pressure of exhaust gases passing therethrough encompasses maintaining the velocity and/or pressure of exhaust gases therethrough, the velocity and/or pressure may be maintained above and/or below a predetermined limit.

The chamber may be configured to reduce the velocity and/or pressure of the exhaust gases passing therethrough. Reducing the velocity of exhaust gases is advantageous as a reduction in the velocity of exhaust gases reduces pressure fluctuations resulting from turbulence, in turn mitigating against damage to an exhaust gas sensor.

The pressure and/or velocity and/or temperature of the exhaust gases passing through the chamber may be modified by any suitable means. For example, there may be one or more vanes disposed within the chamber which redirect the flow of exhaust gases therein. The chamber may be sized to allow the exhaust gases to expand and thereby reduce the pressure and/or velocity of the exhaust gases.

An exhaust gas sensor may be at least partially disposed in the chamber. The exhaust gas sensor may be disposed in the chamber to the degree required to allow it to measure a property of the exhaust gas. As such, in embodiments, only the tip may be disposed within the chamber and the body of the sensor may be located outside of the chamber.

The exhaust gas sensor may be any sensor used to measure a property of the exhaust gas. The exhaust gas sensor may be a temperature sensor, a pressure sensor, an oxygen sensor, or a NOx sensor. Preferably, the exhaust gas sensor is a NOx sensor. The present invention has particular application to exhaust systems in which DEF is injected into the main flow of exhaust gases. The injection of DEF can lead to erroneous readings if the DEF or breakdown products thereof reaches the NOx sensor.

The chamber may be provided at least partially in a recess in a wall of the exhaust gas conduit. As such, the overall cross sectional area of the exhaust gas conduit may be increased relative to the cross-sectional areas immediately upstream and/or immediately downstream of the portion of the exhaust gas conduit including the chamber. This allows the aliquot of exhaust gases which are provided to the chamber to expand and thereby reduce their pressure and/or velocity. The chamber may be defined at least partially by a wall of the recess. Alternatively, an insert which is received in the recess may be provided. By having the chamber disposed in a recess, it is possible to separate an aliquot of the exhaust gases which are passing along the wall of the exhaust gas conduit and allow them to expand into the volume of the recess.

The chamber may be at least partially defined by a wall which separates exhaust gases within the chamber from the remainder of the exhaust gases in the main passage of the exhaust gas conduit. The wall may be in any suitable shape. For example, the wall may be flat or may be curved. The wall may define the chamber by itself or may define the chamber in combination with a wall of a recess. By separating the aliquot of exhaust gases within the chamber from the exhaust gases in the main passage, it is possible for the pressure and/or velocity of the exhaust gases within the chamber to be lower than of the exhaust gases in the main passage. As such, the properties of the exhaust gases within the exhaust gas conduit, particularly the NOx concentration, can be readily measured within the chamber without having to exposure the exhaust gas sensor to the main flow of exhaust gases. The wall may have an upstream edge. The wall may have a downstream edge. The upstream edge of the wall may extend radially inwardly relative to the wall of the main passage.

The chamber may be configured to return the aliquot of exhaust gases to the main flow of exhaust gases. Since it is likely that the exhaust gases will be subject to additional treatments downstream, the aliquot of exhaust gases which is taken off the main flow for measurement may be returned to the main flow. This also allows a continuous flow of exhaust gases through the chamber so that the properties of the exhaust gases can be measured continuously.

The chamber may include an inlet opening and an outlet opening. The inlet opening may be upstream of the outlet opening. The size of the openings can be the same or different. The shape of the openings can be the same or different. The size of the inlet and/or the outlet can be selected to control the amount of exhaust gas entering the chamber as well as the rate at which the exhaust gas is able to leave the chamber. The size of the inlet is selected to provide sufficient exhaust gases to provide an accurate reading, but small enough to ensure that an exhaust gas sensor disposed within the chamber is not overloaded at the maximum flow conditions, since sensors, such as NOx sensors require flow which is slower and has reduced pressure fluctuation than a typical engine outlet exhaust flow. The inlet opening may have a domed shaped edge. In particular, the inlet opening may be at least partially defined by a concave edge. The chamber may be at least partially defined by a concave surface which extends from the concave edge of the inlet opening. The concave surface of the chamber may extend radially into the exhaust gas conduit between around 3 mm to around 11 mm, preferably around 7 mm measured from a wall of the exhaust gas conduit that is radially outwards of the concave surface. The concave surface of the chamber may extend radially into the exhaust gas conduit between around 0.01 to around 0.4 exducer diameters, preferably between around 0.05 to around 0.15 exducer diameters, preferably around 0.1 exducer diameters measured from a wall of the exhaust gas conduit that is radially outwards of the concave surface.

The inlet opening is preferably located upstream of a reducing agent injection point. As such, the exhaust gas conduit may include a reducing agent injection point. The reducing agent injection point is the location at which DEF may be injected into the exhaust gas conduit. The reducing agent injection point may include a reducing agent injector. In order to prevent DEF from interfering with the operation of an exhaust gas sensor, particularly a NOx sensor, it is desirable to sample the exhaust gas from upstream of where DEF may be present.

The inlet opening may be separated from the reducing agent injection point by a distance sufficient such that in operation essentially no reducing agent provided via the reducing agent injection point enters the chamber. If DEF or the decomposition products thereof, particularly ammonia, is mixed with the exhaust gases which are provided to the chamber for analysis, this results in incorrect sensor readings. As such, it is desirable to provide the inlet of the chamber upstream of the point at which DEF is injected at a sufficient distance that none or substantially none of the DEF is recirculated into the chamber.

The inlet opening may be more than about 40 mm, more than about 50 mm, more than about 60 mm, more than about 70 mm, more than about 75 mm, more than about 80 mm, or more than about 90 mm upstream of the reducing agent injection point. The outlet opening may be more than about 5 mm, more than about 10 mm, more than about 12 mm, more than about 15 mm downstream of the reducing agent injection point.

The distance may be the absolute distance between the nearest point of the inlet opening and the reducing agent injection point or may be the linear upstream distance between a plane perpendicular to the direction of flow of exhaust gases associated with the inlet opening and a plane perpendicular to the direction of flow of exhaust gases associated with the reducing agent injection point.

The exhaust gas system may comprise a turbine wheel. The turbine wheel may comprise an exducer defining an exducer diameter. The inlet opening may be spaced upstream of the reducing agent injection point by a distance between around 0.75 to around 1.25 exducer diameters along a centreline of the exhaust gas conduit. The inlet opening may be spaced upstream of the reducing agent injection point by a distance between around 0.9 to around 1.1 exducer diameters along a centreline of the exhaust gas conduit. The inlet opening may be spaced upstream of the reducing agent injection point by a distance between around 1.0 exducer diameters along a centreline of the exhaust gas conduit. The turbine wheel may be at least partially received in the exhaust gas conduit.

As used herein the term “centreline” encompasses a line prescribed by the centroid of a cross-section of the exhaust gas conduit along the direction of flow of exhaust gas. That is to say, the centreline of the exhaust gas conduit is an imaginary line drawn along the exhaust gas conduit which is always positioned at the geometric centre of the exhaust gas flowing therethrough. Typically, although not always, when the exhaust gas conduit is attached to a turbine, the centreline will be an extension of a turbine axis, which may diverge from the turbine axis in dependence upon the geometry of the exhaust gas conduit.

As used herein, the term “exducer” encompasses the part of the turbine wheel configured to discharge exhaust gas to the exhaust gas conduit. The spacing of the chamber inlet opening from the exducer of the turbine wheel may be measured from the most downstream part of the tips of the blades of the turbine wheel to the most upstream part of the inlet opening viewed from the perspective of the centreline.

The inlet opening may be substantially annular. The diameter of the inlet opening may be up to around 0.15 exducer diameters, up to around 0.2 exducer diameters, up to around 0.3 exducer diameters, or up to around 0.35 exducer diameters.

The inlet opening may be spaced apart from the exducer of the turbine wheel by a distance of at most around 3 exducer diameters along a centreline of the exhaust gas conduit. The inlet opening may be spaced apart from the exducer of the turbine wheel by a distance of between around 0 to around 3 exducer diameters along a centreline of the exhaust gas conduit. Preferably, the inlet opening is spaced at a distance greater than around 0 exducer diameters from the turbine wheel along the centreline, because if the inlet opening is spaced at around 0 exducer diameters from the exducer relatively high inefficiencies in the turbine may be observed. The inlet opening may be spaced apart from the exducer of the turbine wheel by a distance of between around 0.75 to around 1.25 exducer diameters along a centreline of the exhaust gas conduit. The inlet opening may be spaced apart from the exducer of the turbine wheel by a distance of between around 0.9 to around 1.1 exducer diameters along a centreline of the exhaust gas conduit. The inlet opening may be spaced apart from the exducer of the turbine wheel by a distance of around 1.0 exducer diameters along a centreline of the exhaust gas conduit. The inlet opening may be spaced apart from the exducer of the turbine wheel by a distance of around 0.8 exducer diameters along a centreline of the exhaust gas conduit. The inlet opening may be spaced apart from the exducer of the turbine wheel by a distance of around 0.35 exducer diameters along a centreline of the exhaust gas conduit.

The chamber may be configured to receive a portion of exhaust gases passing along an interior face of the exhaust gas conduit. The exhaust gas conduit is a pipe that conducts exhaust gases from an internal combustion engine ultimately to the atmosphere. The exhaust gas conduit therefore generally comprises a cylindrical wall, although other cross-sectional shapes are possible and the present invention is not particularly limited to the cross-sectional shape of the exhaust gas conduit. The exhaust gas conduit is suitable for attaching to a turbocharger and for receiving exhaust gases exiting the turbine wheel. It is desirable to minimize the disruption to the flow exiting the turbine wheel and to also minimise flow disruption downstream. By taking an aliquot of exhaust gases passing along an interior face of the exhaust gas conduit, it is possible to minimize disruption to the flow exiting the turbine wheel and to minimize flow disruption downstream. Previously, it was not possible to sample such flow due to the susceptibility of the exhaust gas sensors to exhaust gas temperatures and fluctuations in pressure (pressure pulses) in the exhaust gases, and so exhaust gas sensors were located downstream where the temperatures and magnitude of the fluctuations in pressure were reduced, albeit with the downside that the exhaust gas sensors may be affected by incorrect readings caused by the inclusion of DEF in the exhaust gases being measured.

The exhaust gas conduit may be a diffuser, preferably a turbine diffuser. That is an exhaust gas conduit which is suitable for connection to a turbine housing of a turbocharger or integral with a turbine housing of a turbocharger. Preferably, the exhaust gas conduit is configured to be in direct fluid communication with a turbine housing of a turbocharger. Preferably, exhaust gases leaving the turbine housing are passed directly into the exhaust gas conduit without any intervening units, such as filters or catalysts, although it will be appreciated that the exhaust gas conduit may, in other embodiments be provided downstream of a filter or catalyst. As such, the exhaust gas conduit may include an inlet for receiving exhaust gases from a turbocharger having a turbine wheel, said turbine wheel having a turbo wheel nut plane, wherein the chamber has an inlet disposed from around 20 mm to around 60 mm downstream of the wheel nut plane when in the assembled condition. As mentioned, it is desirable to minimise disruption to the flow of exhaust gases and this may be achieved by providing the inlet opening to the chamber from around 20 mm to around 60 mm from the wheel nut plane. It will be appreciated that this is when the exhaust gas conduit is in the assembled condition with a turbine housing and does not necessarily mean that the inlet opening is from about 20 mm to about 60 mm from an end of the exhaust gas conduit, and the exact placement will depend on how the exhaust gas conduit and the turbine housing are connected to one another. This distance may be readily determined by assembling the exhaust gas conduit and respective turbine housing and measuring the distance between the wheel nut plane and the inlet opening. The distance may be measured to the portion of the inlet opening closest to the wheel nut plane. The distance may be measured between a plane perpendicular to the direction of exhaust flow associated with the upstream end of the inlet opening and the wheel nut plane.

The turbine wheel may comprise an exducer defining an exducer diameter. The exhaust gas conduit may define a centreline and the chamber may comprise an inlet opening spaced apart from the exducer of the turbine wheel by a distance of at most around 3 exducer diameters along the centreline of the exhaust gas conduit. The inlet opening may be spaced apart from the exducer of the turbine wheel by a distance of between around 0 to around 3 exducer diameters along the centreline of the exhaust gas conduit. Preferably, the inlet opening is spaced at a distance greater than around 0 exducer diameters from the turbine wheel along the centreline, because if the inlet opening is spaced at around 0 exducer diameters from the exducer, relatively high inefficiencies in the turbine may be observed. The inlet opening may be spaced apart from the exducer of the turbine wheel by a distance of between around 0.75 to around 1.25 exducer diameters along the centreline of the exhaust gas conduit. The inlet opening may be spaced apart from the exducer of the turbine wheel by a distance of between around 0.9 to around 1.1 exducer diameters along the centreline of the exhaust gas conduit. The inlet opening may be spaced apart from the exducer of the turbine wheel by a distance of around 1.0 exducer diameters along the centreline of the exhaust gas conduit. The inlet opening may be spaced apart from the exducer of the turbine wheel by a distance of around 0.8 exducer diameters along the centreline of the exhaust gas conduit. The inlet opening may be spaced apart from the exducer of the turbine wheel by a distance of around 0.35 exducer diameters along the centreline of the exhaust gas conduit.

The exhaust gas conduit may be an element or component of a turbine housing. The exhaust gas conduit may be integral with the turbine housing. The turbine housing and the exhaust gas conduit may be a single integral component, and thus the ease of manufacturing and assembly is increased. The turbine housing, the exhaust gas conduit and the exhaust gas chamber may be a single integral structure.

The chamber may be an expansion chamber. By allowing the aliquot of exhaust gas to expand in the chamber, its velocity and pressure may be reduced.

The exhaust gas conduit may have an exhaust gas flow axis. The chamber may have an exhaust gas flow axis. The two flow axes may be substantially parallel. Although the exhaust gas may have a swirl component, the overall direction of flow of gas is from an upstream end to a downstream end. By having the chamber substantially parallel to the main passage of the exhaust gas conduit, the main flow of exhaust gases and the flow of the aliquot of exhaust gases within the chamber are substantially parallel. This may assist in minimising disruption to the exhaust flow when it is separated from the main flow and when it is rejoined to the main flow. The exhaust gases within the exhaust gas conduit may rotate as they move through the exhaust gas conduit, particularly where they have previously passed through a turbine wheel of a turbocharger.

The chamber may comprise a chamber centreline. The chamber centreline extends from the centroid of the inlet opening to the centroid of the outlet opening. The chamber centreline may be inclined relative to the centreline of the exhaust gas conduit. The chamber centreline may be inclined at an angle of around 7° relative to the centreline of the exhaust gas conduit, however in alternative embodiments any suitable inclination may be used. For example, chamber centreline may be inclined relative to the centreline of the exhaust gas conduit. The chamber centreline may be inclined at an angle of up to around ±10°, or up to around ±15°, or up to around ±20°. The relative angle between the chamber centreline and the centreline of the exhaust gas conduit may be measured at the centroid of the inlet opening.

The chamber may be at least partially defined by a wall which separates the chamber from the main passage of the exhaust gas conduit. The presence of the chamber, particularly the chamber wall which separates the exhaust gases within the chamber from the exhaust gases within the main passage, protects an exhaust gas sensor disposed within the chamber from this rotating flow of gases. The wall may comprise an upstream edge comprising a lip configured to direct exhaust gases into the chamber. The lip may extend into the main passage of the exhaust gas conduit. The lip may be angled. The lip may be located radially inwardly relative to an adjacent wall of the exhaust gas conduit. In this way, an aliquot of the exhaust gases flowing along the wall of the exhaust gas conduit is separated from the main flow of exhaust gases and directed into the chamber.

The wall may be in the form of a tube having an opening configured to receive an exhaust gas sensor. As such, the chamber may be defined by the tube which is a separate part to the exhaust gas conduit. In this way, the chamber may be formed separately from the rest of the exhaust gas conduit and subsequently assembled. In this way, chambers having different dimensions may be used depending on the predicted requirements.

The exhaust gas conduit may be a two-part exhaust gas conduit. In this way, part of the chamber may be defined by one part and the rest of the chamber may be defined by another part. It will be appreciated that multi-part exhaust gas conduits are also possible. The chamber may be partially defined by a straight machined channel.

The exhaust gas conduit wall may include a receiving element for engaging with a complementary engaging element of an insert. In the assembled form, the exhaust gas conduit wall and the complementary engaging element may define the chamber. The engaging element may comprise a slot and the complementary engaging element may comprise a protrusion configured to engage with the slot. The reverse configuration may alternatively be selected.

The exhaust gas conduit may include a chamber inlet slope. The exhaust gas conduit may include a chamber outlet slope. The exhaust gas conduit may include an inlet slope and an outlet slope. The inlet and/or outlet slopes may be included to facilitate the flow of exhaust gases into and out of the chamber. The inlet and outlet slopes may allow the exhaust gases to take a more linear path into and out of the chamber and thereby reduced turbulence.

The exhaust gas chamber may comprise a plurality of inlet openings. The chamber may comprise an outlet opening. The plurality of inlet openings may be annular inlet openings. The plurality of inlet openings may be circumferentially distributed around a radius of the exhaust gas conduit. The plurality of inlet openings may be equally distributed around a circumferentially extending perimeter of the exhaust gas conduit.

According to a second aspect of the present invention, there is provided a turbine housing including an exhaust gas conduit according to the first aspect of the present invention.

According to a third aspect of the present invention, there is provided a turbocharger including an exhaust gas conduit or a turbine housing according to the first or second aspects of the present invention.

According to a fourth aspect of the present invention, there is provided an internal combustion engine including an exhaust gas conduit, turbine housing, or turbocharger according to the first, second, or third aspects of the present invention.

The inventions according to the second, third, and fourth aspects of the present invention benefit from the advantages of the invention according to the first aspect of the present invention.

According to a fifth aspect of the present invention, there is provided a method of measuring a property of an exhaust gas of an internal combustion engine, the method including the steps of:
a) separating an aliquot of exhaust gases from a main flow of exhaust gases in a exhaust gas conduit into a chamber;
b) modifying the pressure and/or the velocity of the aliquot of exhaust gases; and
c) measuring a property of the aliquot of exhaust gases.

As mentioned, the sensors which are used to measure properties of exhaust gases are sensitive to velocity and pressure fluctuations (pressure pulses), and so by separating an aliquot of exhaust gases from a main flow of exhaust gases, and causing one or both of the velocity and pressure to decrease, it is possible to provide a more hospitable environment to the sensor. In addition, it is possible to measure a property of the exhaust gases further upstream than was previously the case. The chamber also prevents or reduces any reductant, for example, DEF, which is injected into the exhaust gases from interfering with the exhaust gas sensor readings. The chamber may also prevent or reduce other liquids and fluids which are delivered to an exhaust gas stream in an exhaust gas system. By way of example, fluids containing hydrocarbons may be injected into an exhaust gas stream where the exhaust system uses Diesel Oxidation Catalysts (DOC) and/or Diesel Particulate filters (DPF). Exhaust gas sensors, may be sensitive to hydrocarbons, whereby the presence of hydrocarbons contacting the exhaust gas sensor reduces the sensitivity and/or accuracy of the sensor, and may even cause the sensor to fail. Accordingly, use of a chamber as described above may prevent or reduce liquid or gaseous hydrocarbons from contacting the exhaust gas sensor.

The term “aliquot of exhaust gases” encompasses a portion of exhaust gas separated from the main flow, including, but not limited to, a continuous stream of exhaust gas that is separated from the main flow. The term “aliquot of exhaust gases” also encompasses a minor flow or an auxiliary flow of exhaust gas that is separated from the main flow.

Modifying the pressure and/or the velocity of the aliquot of exhaust gases encompasses reducing the pressure and/or velocity of the aliquot of exhaust gases.

The method may further include returning the aliquot of exhaust gases to the main flow of exhaust gases after a property of the aliquot of exhaust gases has been measured. The aliquot of exhaust gases taken for measurement may be returned to the main flow so that it can be treated downstream along with the remainder of the exhaust gases and to allow a continuous flow of exhaust gases through the chamber for measurement.

The aliquot of exhaust gases may be separated from the main flow of exhaust gases passing along an interior face of the exhaust gas conduit. By taking the aliquot of exhaust gases from the exhaust gases passing along an interior face of the exhaust gas conduit, the disruption to the flow of exhaust gases is minimised both upstream and downstream. In addition, the true composition of the exhaust gases can be determined.

The method may include separating the aliquot of exhaust gases from the main flow of exhaust gases at a distance of from about 40 mm to about 90 mm upstream of a reducing agent injection point. As described in respect of the first aspect of the present invention, by taking the aliquot of exhaust gases which is to be measured from upstream of a reducing agent injection point, the exhaust gases are not contaminated by the reducing agent or breakdown products thereof.

The method may include separating the aliquot of exhaust gases from the main flow of exhaust gases at a distance of more than about 40 mm, more than about 50 mm, more than about 60 mm, more than about 70 mm, more than about 75 mm, more than about 80 mm, or more than about 90 mm upstream of the reducing agent injection point.

The main flow of exhaust gas passing through a turbine wheel. The turbine wheel may comprise an exducer defining an exducer diameter. The exhaust gas conduit may define a centreline. The method may include separating the aliquot of exhaust gases from the main flow of exhaust gases at a distance between around 0.75 to around 1.25 exducer diameters along the centreline of the exhaust gas conduit upstream of the reducing agent injection point. The method may include separating the aliquot of exhaust gases from the main flow of exhaust gases at a distance between around 0.9 to around 1.1 exducer diameters along the centreline of the exhaust gas conduit upstream of the reducing agent injection point. The method may include separating the aliquot of exhaust gases from the main flow of exhaust gases at a distance around 1.0 exducer diameters along a centreline of the exhaust gas conduit upstream of the reducing agent injection point. The turbine wheel may be at least partially received in the exhaust gas conduit.

The method may include the main flow of exhaust gas may passing through a turbine wheel. The turbine wheel may comprise an exducer defining an exducer diameter. The method may include separating the aliquot of exhaust gases from the main flow of exhaust gases at a distance of at most around 3 exducer diameters along a centreline of the exhaust gas conduit from the exducer. The method may include separating the aliquot of exhaust gases from the main flow of exhaust gases at a distance of between around 0 to around 3 exducer diameters along a centreline of the exhaust gas conduit from the exducer. Preferably, the aliquot of exhaust gases is separated from the main flow at a distance greater than around 0 exducer diameters from the turbine wheel along the centreline, because if the inlet opening is spaced at around 0 exducer diameters from the exducer relatively high inefficiencies in the turbine may be observed. The method may include separating the aliquot of exhaust gases from the main flow of exhaust gases at a distance of between around 0.75 to around 1.25 exducer diameters along a centreline of the exhaust gas conduit from the exducer. The method may include separating the aliquot of exhaust gases from the main flow of exhaust gases at a distance of between around 0.9 to around 1.1 exducer diameters along a centreline of the exhaust gas conduit from the exducer. The method may include separating the aliquot of exhaust gases from the main flow of exhaust gases at a distance of around 1.0 exducer diameters along a centreline of the exhaust gas conduit from the exducer. The method may include separating the aliquot of exhaust gases from the main flow of exhaust gases at a distance of around 0.8 exducer diameters along a centreline of the exhaust gas conduit from the exducer. The method may include separating the aliquot of exhaust gases from the main flow of exhaust gases at a distance of around 0.35 exducer diameters along a centreline of the exhaust gas conduit from the exducer. Said distance may all be upstream of a reducing agent injection point. As described in respect of the first aspect of the present invention, by taking the aliquot of exhaust gases from upstream of a reducing agent injection point, the exhaust gases are not contaminated by the reducing agent or breakdown products thereof. The turbine wheel may be at least partially received in the exhaust gas conduit. The internal combustion engine may include a turbocharger having a turbine wheel and a turbo wheel nut plane, wherein the method further includes separating the aliquot of exhaust gases from the main flow of exhaust gases at a distance of from around 20 mm to around 60 mm of the turbine wheel nut plane.

The internal combustion engine may include a turbocharger having a turbine wheel. The turbine wheel may comprise an exducer defining an exducer diameter. The exhaust gas conduit may define a centreline. The method may further include separating the aliquot of exhaust gases from the main flow of exhaust gases at a distance of at most around 3 exducer diameters along the centreline of the exhaust gas conduit from the exducer. The method may include separating the aliquot of exhaust gases from the main flow of exhaust gases at a distance of between around 0 to around 3 exducer diameters along the centreline of the exhaust gas conduit from the exducer. Preferably, the aliquot of exhaust gases is separated from the main flow at a distance greater than around 0 exducer diameters from the turbine wheel along the centreline, because if the inlet opening is spaced at around 0 exducer diameters from the exducer relatively high inefficiencies in the turbine may be observed. The method may include separating the aliquot of exhaust gases from the main flow of exhaust gases at a distance of between around 0.75 to around 1.25 exducer diameters along the centreline of the exhaust gas conduit from the exducer. The method may include separating the aliquot of exhaust gases from the main flow of exhaust gases at a distance of between around 0.9 to around 1.1 exducer diameters along the centreline of the exhaust gas conduit from the exducer. The method may include separating the aliquot of exhaust gases from the main flow of exhaust gases at a distance of around 1.0 exducer diameters along the centreline of the exhaust gas conduit from the exducer. The method may include separating the aliquot of exhaust gases from the main flow of exhaust gases at a distance of around 0.8 exducer diameters along the centreline of the exhaust gas conduit from the exducer. The method may include separating the aliquot of exhaust gases from the main flow of exhaust gases at a distance of around 0.35 exducer diameters along the centreline of the exhaust gas conduit from the exducer. Again, as described in respect of the first aspect of the present invention, the present invention allows the exhaust gas to be measured further upstream than would otherwise be possible and also minimises disruption to the flow of exhaust.

Any property of the exhaust gas may be measured. For example, the temperature, pressure, or composition, such as NOx concentration of oxygen concentration, may be measured. Preferably, the NOx concentration is measured. Although the invention is not necessarily limited to any particular property or sensor, the present has particular application to NOx sensors in apparatuses which include the injection of DEF into the main flow of exhaust gases.

According to a sixth aspect of the present invention, there is provided an exhaust gas conduit for an exhaust system of internal combustion engine, the exhaust gas conduit including a ramp and a wall at least partially surrounding an opening configured to receive an exhaust gas sensor, the ramp and wall being configured to define a volume for receiving an exhaust gas sensor and to shield an exhaust gas sensor from a main flow of exhaust gases passing through the exhaust gas conduit.

As mentioned, exhaust gas sensors can be damaged by high exhaust gas velocities and pressure fluctuations (pressure pulses). It is also desirable to provide exhaust gas sensors further upstream than has previously been possible. The invention according to the sixth aspect of the present invention allows an exhaust gas sensor to be provided further upstream than has previously been possible by providing a ramp. The ramp deflects exhaust gases past the exhaust gas sensor and thereby protects the sensor from direct interaction with the exhaust gases. The wall also protects the exhaust gas sensor from high velocity exhaust gases and/or the pressure fluctuations above the operating parameters of the sensor. Even through the ramp deflects the flow of exhaust gases, an area of lower pressure is generated downstream of the ramp and the wall which allows exhaust gases to spill into the area of an exhaust gas sensor where they can be measured. Thus, an exhaust gas sensor provided in such a exhaust gas conduit is not subject to the direct flow of exhaust gases, but is still able to measure a property of the exhaust gases.

The ramp may slope radially inwardly from an upstream end to a downstream end. As such, ramp increases in height from the upstream end to the downstream end. This causes the flow of exhaust gases to be deflected radially inwardly.

The ramp may be continuous with the wall.

The wall may have at least one discontinuity. The discontinuity may be disposed in a downstream portion of the wall. The discontinuity may be configured to allow an aliquot of exhaust gases to spill into the volume defined by the ramp and the wall. By having a discontinuity at a downstream end of the wall, exhaust gases are able to pass into the volume where an exhaust gas sensor may be located. Such gases are at a lower pressure and/or a lower velocity than the main flow of exhaust gases.

The exhaust gas conduit may include an exhaust gas sensor received in the opening. The exhaust gas sensor may therefore be provided in the volume defined by the ramp and the wall. Any suitable exhaust gas sensor may be included. Preferably, the exhaust gas sensor is a NOx sensor.

The wall and the ramp may extend into the exhaust gas conduit by an equal or greater extent than the exhaust gas sensor. Since it is desired to avoid the exhaust gas sensor being exposed directly to the flow of exhaust gases within the exhaust gas conduit, it is preferable that the exhaust gas sensor is inset relative to the ramp and the wall.

The wall may be substantially conformal to the shape of the exhaust gas sensor, and, in particular, to the cross-sectional shape of the exhaust gas sensor. When the wall is substantially conformal to the shape of the exhaust gas sensor, the size of the volume for receiving the exhaust gas sensor is reduced, thus providing tighter and thereby improved shielding of the exhaust gas sensor.

According to a seventh aspect of the invention, there is provided a turbine comprising the exhaust gas conduit of the sixth aspect of the invention. Optionally, the exhaust gas conduit may be defined by a turbine outlet passage of the turbine, and moreover by a diffuser defining at least part of such a turbine outlet passage (i.e. such that the ramp and wall are positioned within the diffuser). In such embodiments, the ramp and wall are located in relatively close proximity to an outlet of a turbine wheel of the turbine.

According to an eighth aspect of the invention, there is provided a turbocharger comprising the turbine of the seventh aspect of the invention.

According to a ninth aspect of the present invention, there is provided a shield for an exhaust gas sensor, said shield configured to substantially surround an exhaust gas sensor tip.

Again, in order to protect an exhaust gas sensor from the exhaust gases and allow the sensor to be provided further upstream than has previously been possible, the provision of a shield which substantially surrounds an exhaust gas sensor tip allows the exhaust gas sensor to be provided further upstream than would otherwise be possible. The shield may also be configured to control the amount of exhaust gas provided to the exhaust gas sensor tip.

The shield may be configured to be attached to an exhaust gas sensor or to a boss configured to receive an exhaust gas sensor.

The shield may include a plurality of protrusions disposed radially around a perimeter of the shield.

The shield may include a plurality of apertures in a sidewall thereof. The plurality of apertures may be associated with respective protrusions disposed radially around a perimeter of the shield.

The optional and/or preferred features for each aspect of the invention set out herein are also applicable to any other aspects of the invention. The various features of the aspects of the present invention may be combined with features of other aspects of the present invention in any combination, except where the features are mutually incompatible.

### DETAILED DESCRIPTION

Specific embodiments of the present invention will now be described, by way of example only, with reference to the accompanying drawings in which:

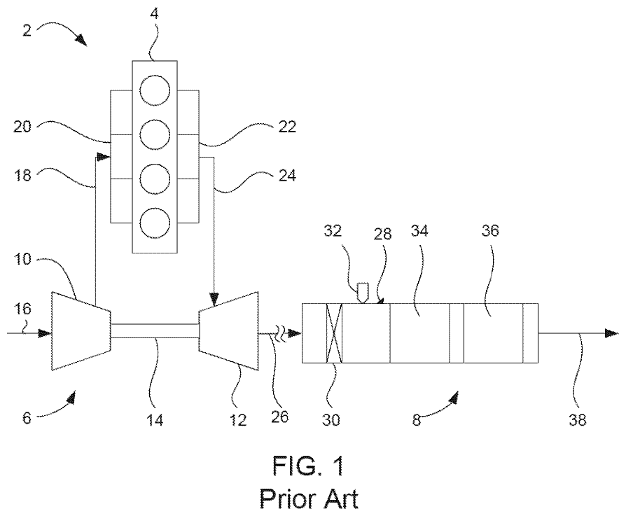

FIG. 1 is a schematic view of a known turbocharged diesel engine system;

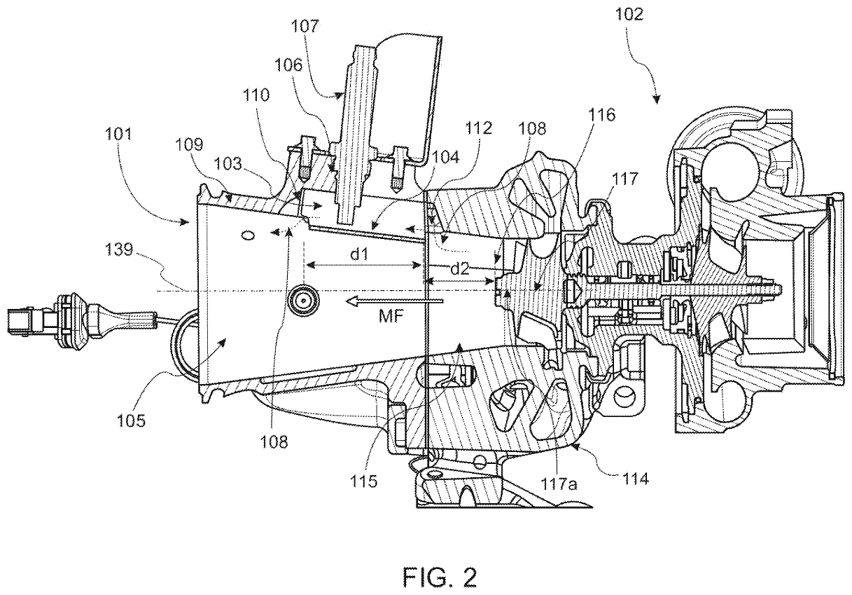

FIG. 2 is a longitudinal cross-section of an exhaust gas conduit according to a first aspect of the present invention as attached to a turbocharger;

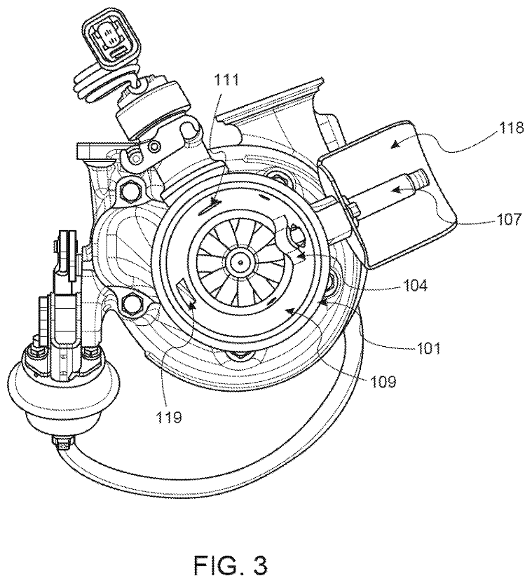

FIG. 3 is a lateral cross-section of an exhaust gas conduit according to a first aspect of the present invention as attached to a turbocharger;

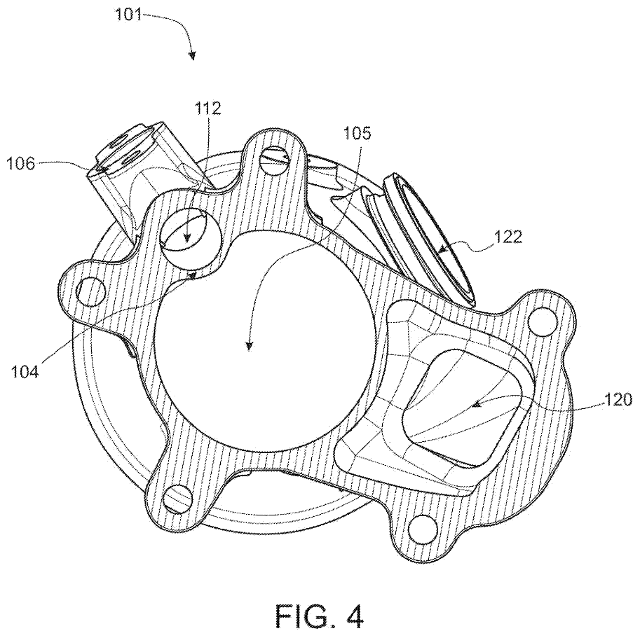

FIG. 4 is a depiction of an exhaust gas conduit according to a first aspect of the present invention viewed from an upstream end towards a downstream end;

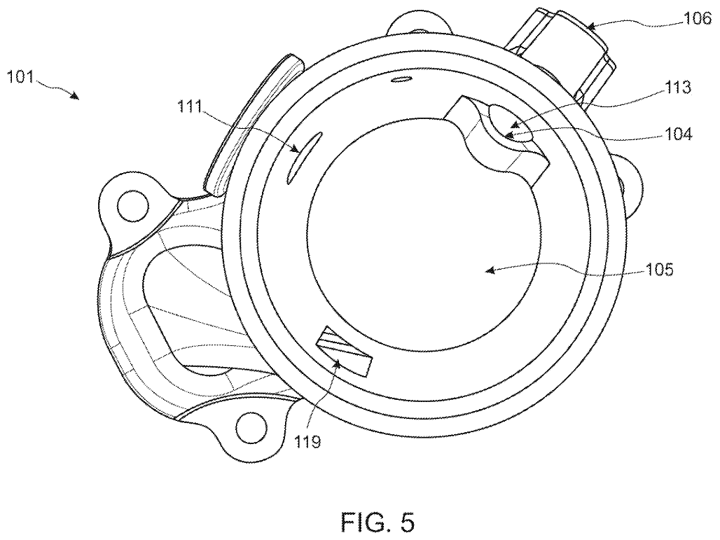

FIG. 5 is a depiction of an exhaust gas conduit according to a first aspect of the present invention viewed from a downstream end towards an upstream end;

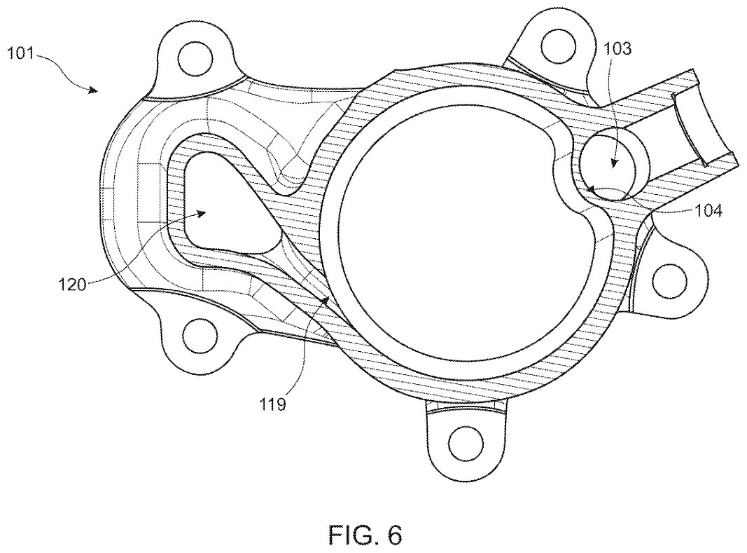

FIG. 6 is a lateral cross-section through an exhaust gas conduit according to a first aspect of the present invention;

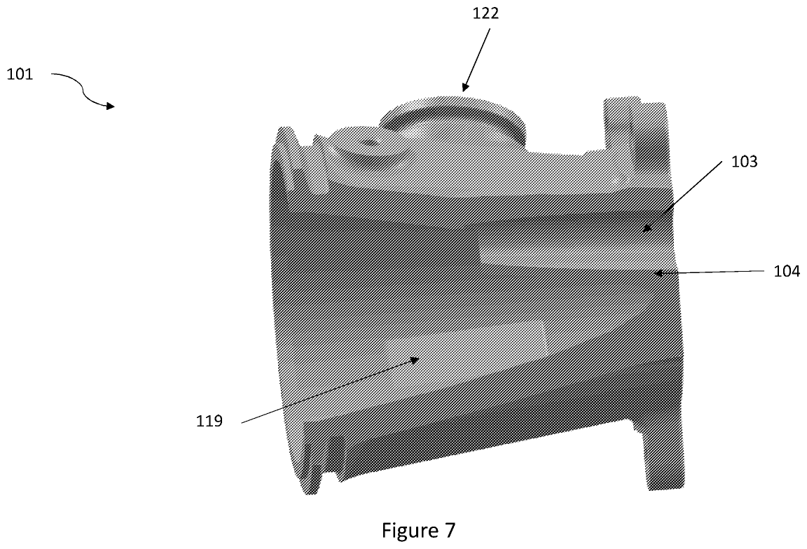

FIG. 7 is a longitudinal cross section through an exhaust gas conduit according to the first aspect of the present invention;

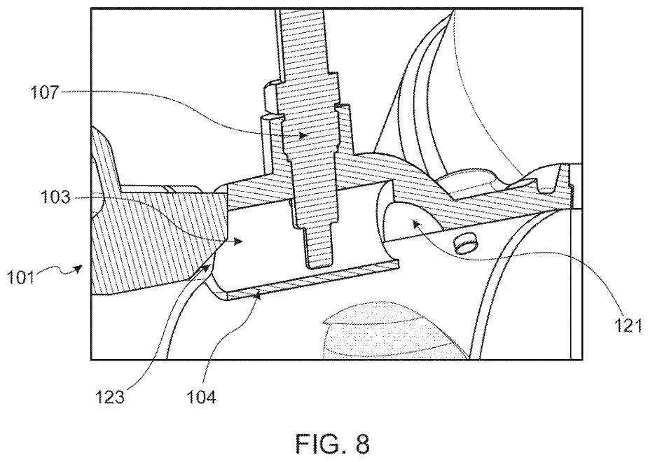

FIG. 8 is a longitudinal cross-section through an exhaust gas conduit according to a first aspect of the present invention showing an exhaust gas sensor in situ;

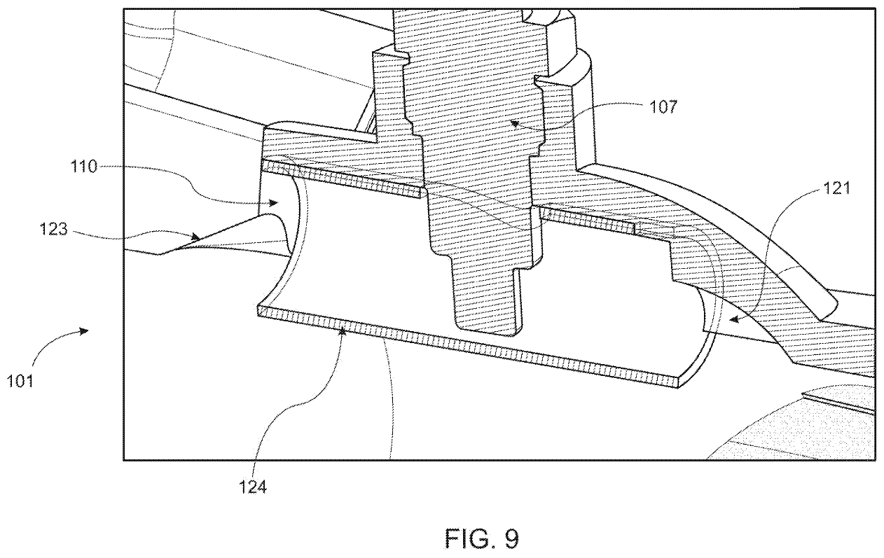

FIG. 9 is a longitudinal cross-section through an exhaust gas conduit according to a first aspect of the present invention;

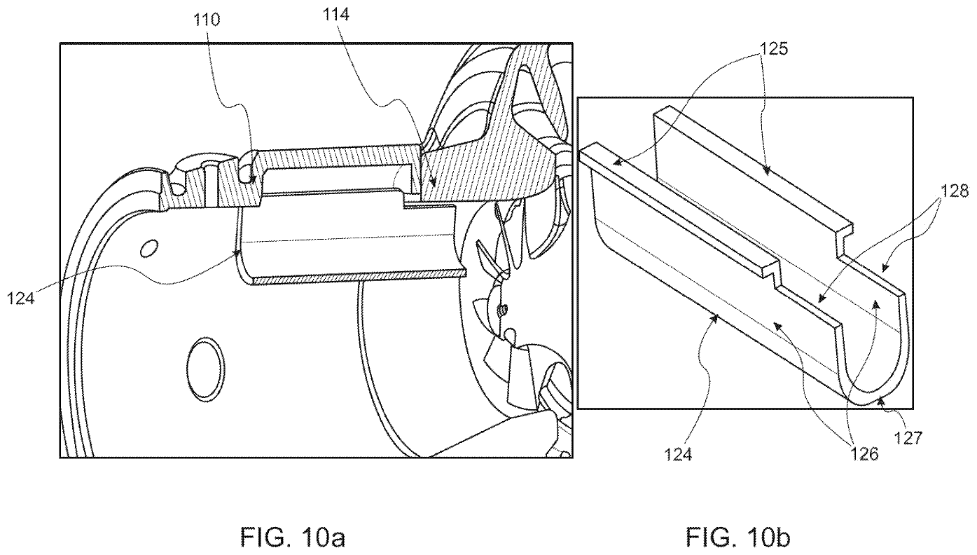

FIGS. 10a and 10b are a longitudinal cross-section through an exhaust gas conduit according to a first aspect of the present invention and a depiction of an insert;

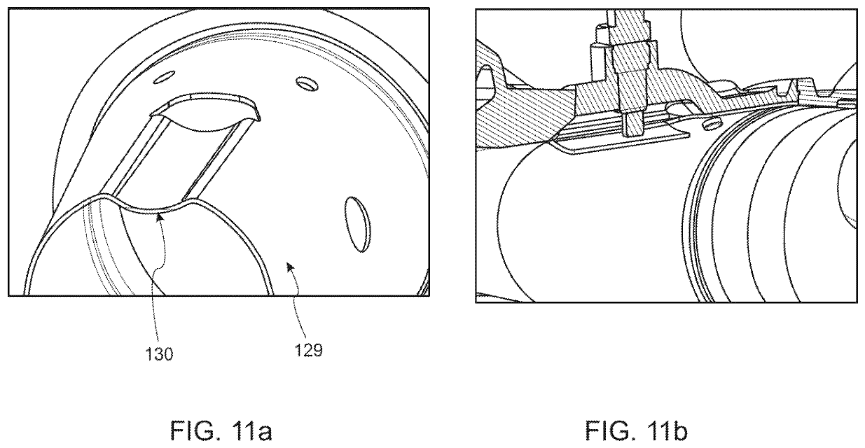

FIGS. 11a and 11b are depictions of an exhaust gas conduit according to a first aspect of the present invention;

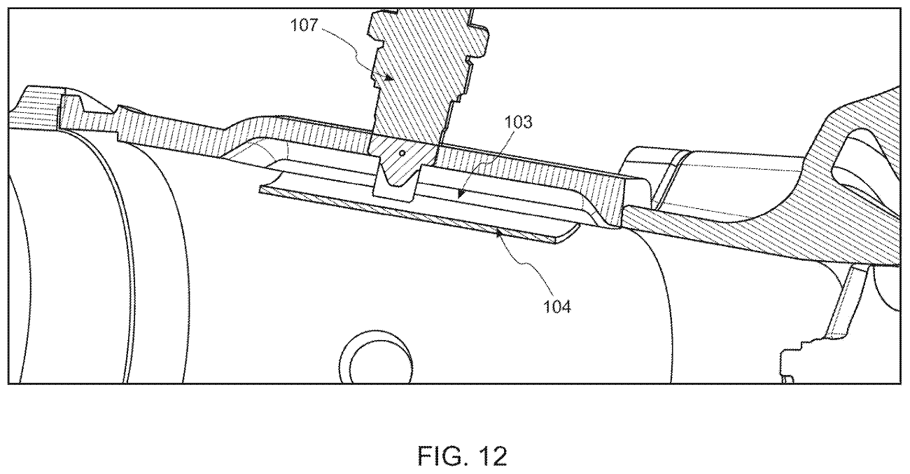

FIG. 12 is a cross-section of an exhaust as conduit according to the first aspect of the present invention;

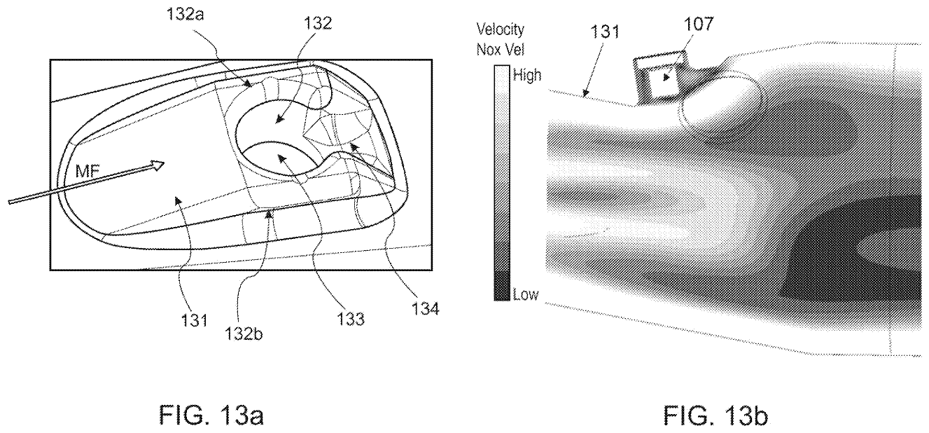

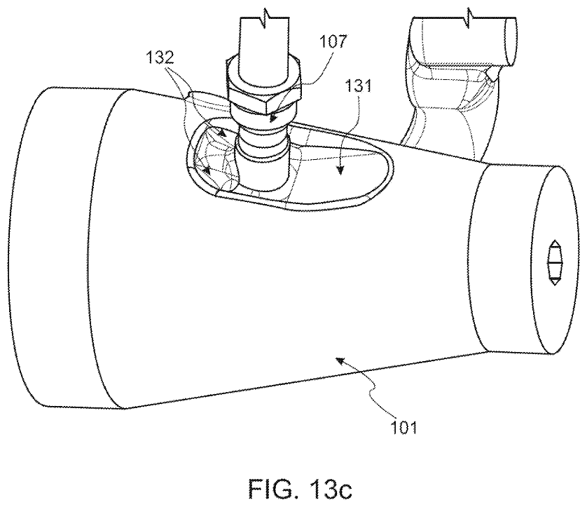

FIGS. 13a, 13b and 13c are a schematic depiction of an exhaust gas conduit according to the sixth aspect of the present invention, and a depiction of the flow velocity of exhaust gases over a ramp, and a depiction of an exhaust gas conduit comprising such a ramp, respectively;

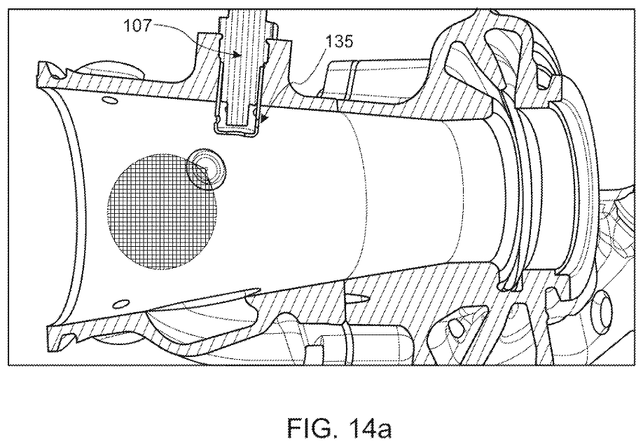

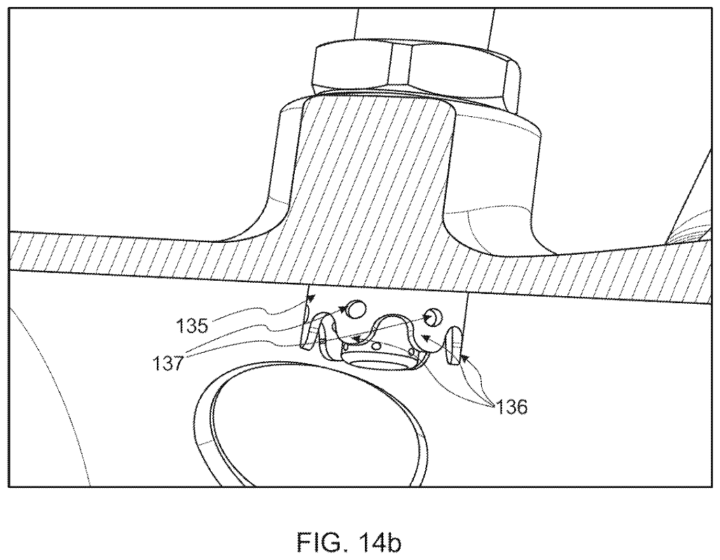

FIGS. 14a and 14b are depictions of a cross-section through a turbocharger and associated exhaust conduit including a shield according to the seventh aspect of the present invention, and of a shield according to the seventh aspect of the present invention;

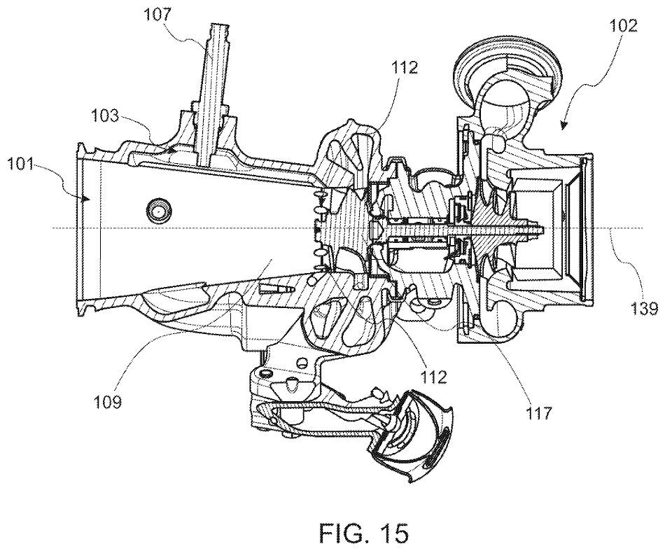

FIG. 15 is a longitudinal cross-section of an exhaust gas conduit according to a first aspect of the present invention as attached to a turbocharger;

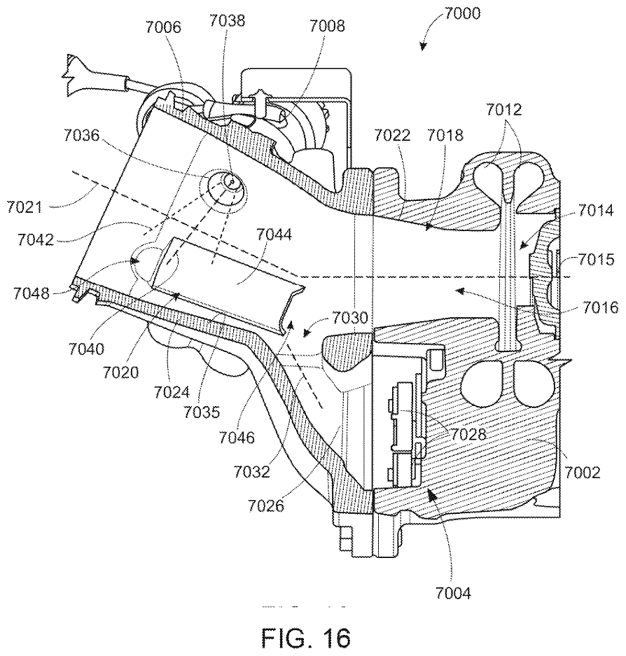

FIG. 16 is a cross-sectional plan view of a turbine in accordance with one or more aspects of the present invention;

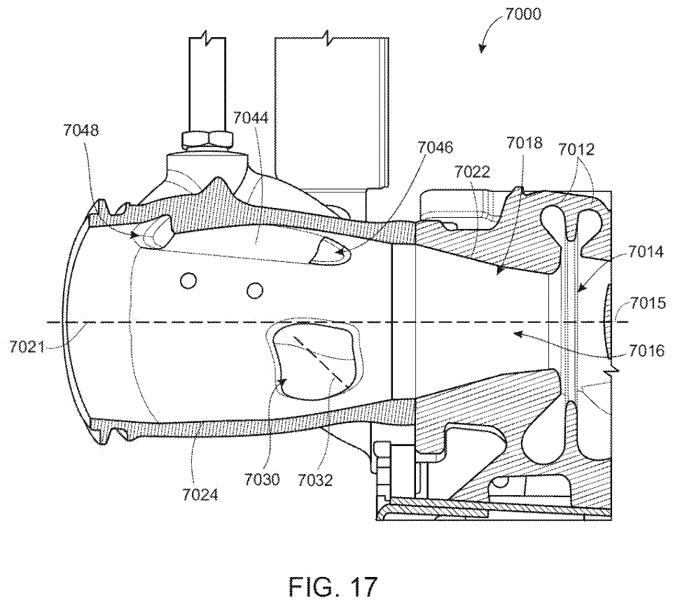

FIG. 17 is a cross-sectional top view of the turbine of FIG. 16;

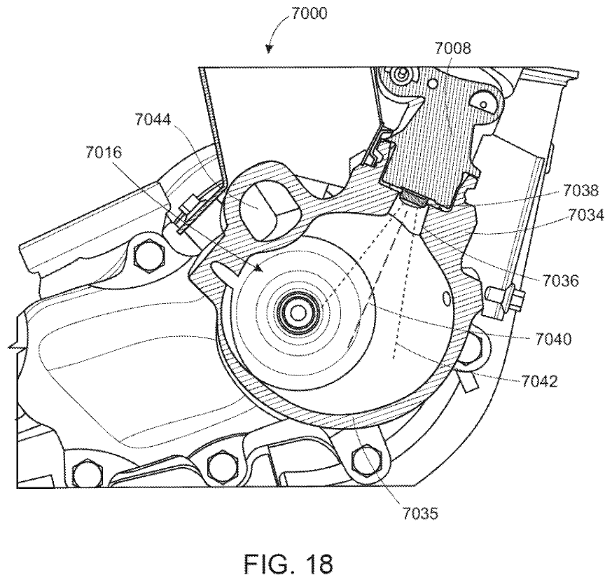

FIG. 18 is a cross-sectional end view of the turbine of FIG. 16 taken through the position of the dosing module;

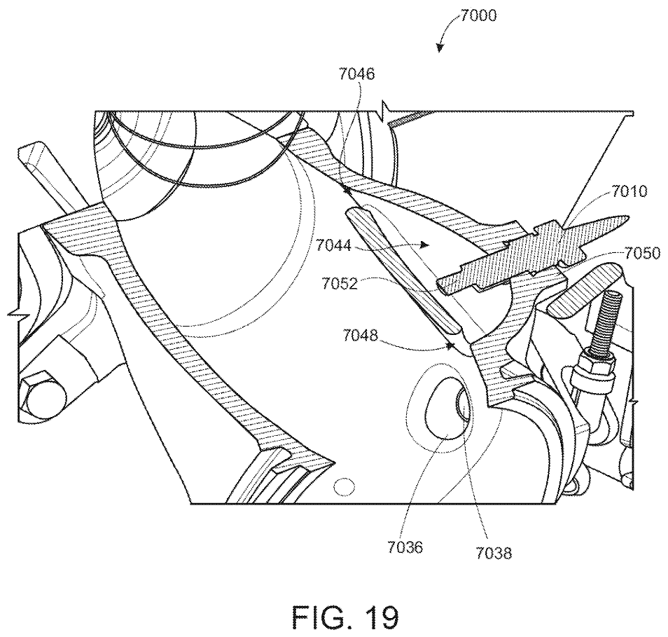

FIG. 19 is a cross-sectional side view of the turbine of FIG. 16 taken through the sensing passage;

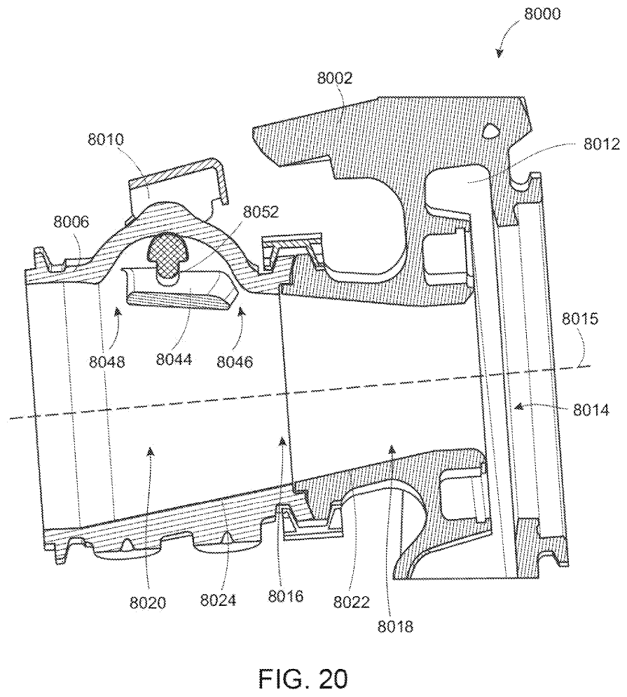

FIG. 20 is a cross-sectional side view of a turbine in accordance with one or more aspects of the present invention;

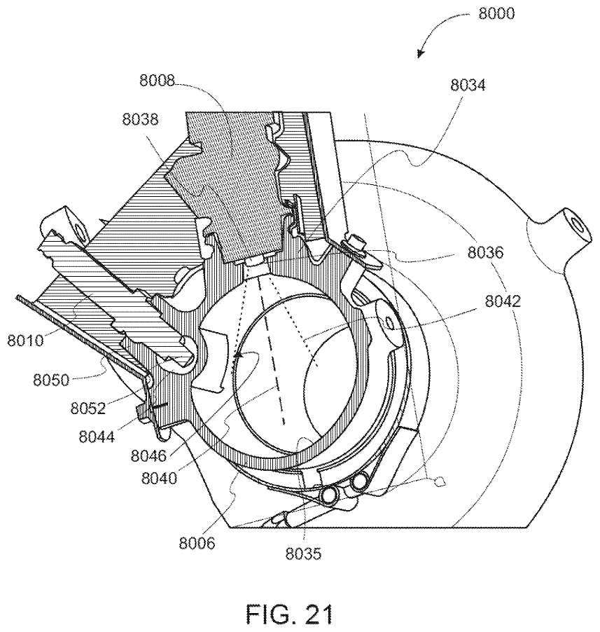

FIG. 21 is a cross-sectional end view of the turbine of FIG. 20 taken through the position of the dosing module and sensing passage; and

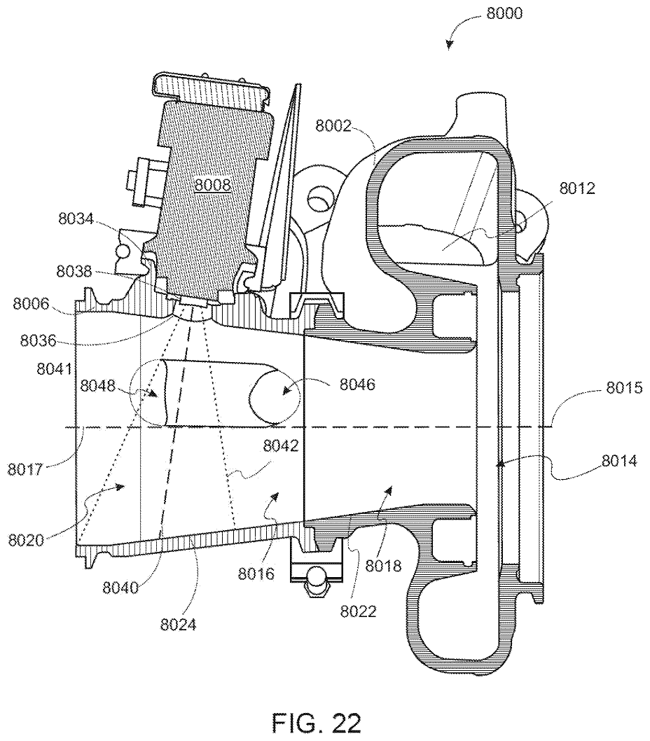

FIG. 22 is a cross-sectional side view of the turbine of FIG. 20 taken through the dosing module.

FIG. 1 shows a schematic view of a turbocharged diesel engine system 2 according to the prior art. The system 2 comprises a diesel internal combustion engine 4, a turbocharger 6 and an exhaust gas aftertreatment system 8. The turbocharger 6 comprises a compressor 10 and a turbine 12 mounted to a common turbocharger shaft 14 so that the two rotate in unison. The compressor 10 receives intake air from a low pressure intake duct 16 connected to atmosphere. The low pressure intake duct 16 may comprise a particulate filter to clean the intake air. The compressor 10 compresses the intake air using power provided by the turbocharger shaft 14 and supplies the compressed intake air to the engine 4 via a high pressure intake duct 18 and an intake manifold 20. Although not shown, the high pressure intake duct 18 may comprise an intercooler configured to cool the intake air before it reaches the engine 4. Inside the engine 4, an internal combustion process takes place and useful work is produced. As a result of the internal combustion process, exhaust gases are created by the engine 4. The engine 4 is fluidly connected to an exhaust manifold 22 which is in turn connected to the turbine 12 via a high pressure exhaust gas duct 24. The turbine 12 extracts energy from the exhaust gas to drive the turbocharger shaft 14 and thereby power the compressor 10. Exhaust gas leaving the turbocharger 12 is supplied to the exhaust gas aftertreatment system 8 via a downpipe 26. The downpipe 26 is relatively long in extent, for example at least 2 metres in length, as indicated by the broken line in FIG. 1.

The exhaust gas aftertreatment system 8 comprises a decomposition chamber 28 having a diameter larger than that of the downpipe 26. The decomposition chamber 28 comprises a mixing element 30 disposed therein. The mixing element 30 typically comprises a number of baffles configured to deflect the flow through the decomposition chamber 28 to cause turbulence within the decomposition chamber 28. The exhaust gas aftertreatment system 8 comprises a dosing module 32 configured to inject an exhaust gas aftertreatment fluid, and specifically Diesel Exhaust Fluid (DEF), into the decomposition chamber 28 downstream of the mixing element 30 in the region where the exhaust gas is most turbulent. Heat exchange between the DEF and the exhaust gas within the decomposition chamber 28 causes the urea contained within the DEF to decompose into the reductants ammonia (NH₃) and Isocyanic Acid (HNCO). The mixture of reductants and exhaust gas is then passed to a selective catalytic reducer (SCR) 34 and a diesel oxidation catalyst (DOC) 36. Finally, the exhaust gas is passed to an outlet duct 38 and onwards to a muffler (not shown) before being discharged to atmosphere.

The focus of the present invention is the incorporation into an exhaust gas conduit of a separate chamber which houses an exhaust gas sensor and which is configured to reduce the velocity and/or pressure fluctuations within the chamber to extend the lifetime of the sensor and/or to allow the sensor to be located further upstream than previously possible.

FIG. 2 is a longitudinal cross-section of an exhaust gas conduit 101 according to a first aspect of the present invention as attached to a turbocharger 102. It will be appreciated that the features depicted in FIG. 2 (as well as the other figures) which do not comprise the exhaust gas conduit 101 are for context and the specific disclosure of such figures does not necessarily limit the scope of the invention to requiring each of the depicted features. The exhaust gas conduit 101 includes a chamber 103, which may also be referred to as an exhaust gas sensor channel. The chamber 103 includes a chamber wall 104. The chamber wall 104 at least partially defines the chamber 103. The chamber wall 104 is configured to separate an aliquot of exhaust gases 108 from the main flow MF of exhaust gases. The chamber wall 104 is radially inset from the main wall 109 of the exhaust gas conduit 101 to allow the aliquot of exhaust gas 108 which is to be measured to be split off from the main flow MF of exhaust gases. The chamber 103 is configured to allow the aliquot of exhaust gases 108 to expand therein. The chamber 103 includes a mounting point 106 for mounting an exhaust gas sensor 107. The mounting point 106 can be configured to receive and retain an exhaust gas sensor 107 by any suitable means, such as, for example a screw thread. The mounting point 106 is configured to retain an exhaust gas sensor 107 therein to allow the portion of the exhaust gas sensor 107 which is operable to measure a property of exhaust gas to be exposed to any exhaust gases within the chamber 103. The chamber 103 is partially defined by a recess 110 in the main wall 109 of the exhaust gas conduit. The recess 110 provides additional volume into which the aliquot of exhaust gases 108 may expand.

The exhaust gas conduit 101 may include a reducing agent injection point 111 through which reducing agent, may be injected via an appropriate injector or doser. The reducing agent may include, but is not limited to, DEF. The chamber 103 includes an inlet opening 112 through which exhaust gases can enter the chamber 103 and an outlet opening 113 through which exhaust gases can leave the chamber 103 and enter the main passage 105 and re-join the main flow MF of exhaust gases. The inlet opening 112 is upstream of the reducing agent injection point 111 so that the reducing agent or breakdown products thereof is unable to enter the chamber 103 via the inlet opening 112, thereby ensuring that the readings of the exhaust gas sensor 107 within the chamber 103 are not affected by the presence of reducing agent thereby providing incorrect readings. The chamber wall 104 serves to block reducing agent from entering the chamber 103. The distance d1 between a plane corresponding to a mid-point of the reducing agent injection point 111 and a plane corresponding to the inlet opening 112 is selected to prevent the entry of reducing agents injected via the reducing agent injection point 111 from entering the chamber 103.

The exhaust gas conduit 101 include a main passage 105 through which the main flow of exhaust gases MF passes in operation. In the depicted embodiment, the main passage 105 is frustoconical in shape and the cross-sectional area of the main passage 105 increase in the downstream direction. Where the exhaust gas conduit 101 has such a frustoconical shape, it may be referred to as a diffuser and where it receives exhaust gases from a turbine end of a turbocharger, it may be referred to as a turbine diffuser. The exhaust gas conduit 101 may be integral with a turbine housing 114 or may be a separate component (as depicted in FIG. 2). Where the exhaust gas conduit 101 is a separate component, it may have suitable means, such as via one or more bolts, for affixing the exhaust gas conduit 101 to a turbine housing 114, although the present invention is not particularly limited by any particular affixing means. The exhaust gas conduit 101 is configured such that in the assembled condition the inlet opening 112 is at a particular distance d2 downstream of a wheel nut plane that the separation of the aliquot of exhaust gases 108 does not adversely affect the flow of exhaust gases leaving the turbine. As depicted, the inlet opening 112 is located at an end of the exhaust turbine conduit 101 since the turbine housing 114 includes its own exhaust conduit portion 115 which provides the required distance d2 between the turbo wheel nut plane 116 and the inlet opening 112. In embodiments where there is no or too short a turbine housing exhaust conduit portion 115, the inlet opening 112 of the exhaust gas conduit 101 may be located downstream of the end of the exhaust gas conduit 101 to provide the desired distance d2 between the inlet opening 112 and the turbo wheel nut plane 116 in the assembled condition. The sizes of the inlet opening 112 and the outlet opening 113 are selected such that the exhaust gas sensor 107 is provided with sufficient exhaust gases to be able to measure a property of the exhaust gases and is not provided with too great an amount of exhaust gases at peak exhaust flow rates.

In operation, exhaust gas is expanded via the turbine wheel 117 causing the turbine wheel 117 to rotate. The turbine wheel comprises an exducer 117a defining an exducer diameter. The turbine wheel 117 is attached to a shaft and the shaft is attached to a compressor wheel. The rotation of the turbine wheel 117 is transferred to the compressor wheel via the common shaft, which is thereby caused to rotate. The rotation of the compressor wheel compresses air, which is fed to an internal combustion engine. The exhaust gases leaving the turbine wheel 117 pass into the main passage 105 of the exhaust gas conduit 101 and an aliquot of exhaust gases 108 of the exhaust gases leaving the turbine wheel 117 and travelling along a wall of the exhaust gas conduit 101 and/or the exhaust conduit portion 115 of the turbine housing 114 is separated and passed into the chamber 103 via inlet opening 112. The aliquot of exhaust gases 108 which enter the chamber 103 expands and thereby reduces its velocity and/or pressure. The pressure of the exhaust gases is not consistent and there are pressure pulses therein corresponding to the combustion cycle of the cylinders of the internal combustion engine to which the exhaust gas conduit 101 is attached. The expansion of the aliquot of exhaust gases 108 within the chamber 103 reduces the magnitude of the pressure pulses and gas velocity and thereby provides a more appropriate environment for an exhaust gas sensor 107 within the chamber 103, thereby allowing the exhaust gas sensor 107 to be positioned further upstream than would otherwise be the case. In order to meet emissions requirements, a reducing agent, which may include DEF, is injected into the main flow MF of the exhaust gases. The reducing agent is injected downstream of the inlet opening 112 of the chamber 103 and so none of the reducing agent is able to enter the chamber 103 to affect the reading of the exhaust gas sensor 107, which is preferably a NOx sensor. The chamber wall 104 prevents reducing agent from entering the chamber 103. The exhaust gas sensor 107 is able to measure a property of the aliquot of exhaust gases 108. The measurement of the property can then be used to determine an operating parameter of the engine or exhaust system. The aliquot of exhaust gases 108 is then returned to the main flow MF of exhaust gases.

In the embodiment shown, the inlet opening 112 of the chamber 103 is spaced apart from the exducer 117a by around 1 exducer diameters along the centreline 139 of the exhaust gas conduit 101. In other embodiments, the inlet opening 112 may be spaced apart from the exducer 117a by between around 0 to around 3, between around 0.75 to around 1.25, between around 0.9 to around 1.1, 0.35 or 0.8 exducer diameters along the centreline 139.

The distances from the turbine exducer 117a to the inlet opening 112 may be measured from the tips of the blades of the turbine wheel 117 to the centroids of the inlet opening 112. In some embodiments, the exhaust gas conduit 101 may define a non-linear path comprising bends. In such instances, the distances from the turbine exducer 117a to the inlet opening 112 may be measured along a centerline of the of the exhaust gas conduit 101. The centerline is the line prescribed by the centroid of the exhaust gas conduit 112 along the direction of the main flow.

FIG. 3 is a lateral cross-section of an exhaust gas conduit 101 as attached to a turbocharger. The chamber wall 104 protrudes into the main passage 105 in order to divert an aliquot of exhaust gases 108 into the chamber 103. A tip of an exhaust gas sensor 107 is provided in the chamber 103 for measuring a property of exhaust gases therein. An optional exhaust gas sensor shield 118 may be provided. An optional wastegate passage outlet 119 is included in the main wall of the exhaust gas conduit 109. The chamber wall 104 is depicted as being curved, but it will be appreciated that the chamber wall may be linear. The chamber wall 104 is depicted as having fillets to provide a smooth transition and to minimise disruption to exhaust gases which are flowing in a spiral down the exhaust gas conduit 101.

FIG. 4 depicts an exhaust gas conduit 101 viewed from an upstream end towards a downstream end. The inlet opening 112 to the chamber 113 is circular in cross-section, although it will be appreciated that the cross-sectional shape may be a shape other than circular. The chamber wall 104 protrudes into the main passage 105. The chamber wall 104 is curved in the radial direction. In other embodiments, the chamber wall 104 may be flat. In other embodiments, the chamber wall 104 may comprise at least one portion which is curved and include at least one portion which is flat, the at least one curved portion may be curved in the radial direction. In other embodiments, the chamber 104 may comprise a plurality of curved portions and a plurality flat portions; at least one of the plurality of curved portions may be curved in the radial direction. A curved portion, may include a bump, a dome, a protrusion, or a recess. The chamber wall 104 may be substantially flat and comprise one curved portion. The exhaust gas conduit 101 includes a reducing agent injector mount 122 configured to receive a reducing agent injector or doser (not shown).

FIG. 5 depicts an exhaust gas conduit 101 viewed from a downstream end towards an upstream end. The outlet opening 113 of the chamber 103 has a different shape than the inlet opening 112 as the outlet opening 113 includes a chamber outlet slope 121 configured to direct exhaust gases from within the chamber 103 into the main flow MF of exhaust gases within the exhaust gas conduit 101.

FIG. 6 is a lateral cross section through an exhaust gas conduit 101 showing the circular cross-section of the chamber 103. The optional wastegate passage 120 and wastegate passage outlet 119, although it will be appreciated that these are not necessarily included in an exhaust gas conduit according to the present invention. The optional wastegate passage 120 and wastegate passage outlet 119 may be provided where the exhaust gas conduit is used alongside a turbocharger system.

FIG. 7 is a further depiction of an exhaust gas conduit 101 showing the chamber 103, the chamber 104 as well as the reducing agent injector mount 122, and the optional wastegate passage outlet 119.

FIG. 8 is another depiction of an exhaust gas conduit 101 including a chamber 103 ad an exhaust gas sensor 107. The chamber wall 104 is linear in the longitudinal direction. The chamber 103 includes a chamber inlet slope 123 and a chamber outlet slope 121, which assist in directing the flow of the aliquot of exhaust gases 108 passing through the chamber 103.

FIG. 9 shows an embodiment of an exhaust gas conduit 101 in which the chamber 103 is defined by a chamber insert 124. The chamber insert 124 comprises a tube that is received in a corresponding recess 110 of the exhaust gas conduit 101. In the previously depicted embodiments, the chamber 103 is defined by the chamber wall 104 and a wall of the recess 110, and such configurations can be produced by common machining methods, by additive manufacturing, or a combination thereof. By providing a chamber insert 124 to define the chamber 103, it is possible to select an appropriate chamber shape and dimension depending on the desired characteristics.

FIGS. 10a and 10 b shows an embodiment of an exhaust gas conduit 101 in which the chamber 103 is defined by a chamber insert 124. In this embodiment, the chamber insert 124 does not comprise a tube with a continuous wall like the embodiment of FIG. 9, but defines a portion of the chamber 103 along with a wall of the recess 110. The chamber insert 124 comprises a U-shaped component. The chamber insert 124 includes engaging elements in the form of protrusions 125 which run along a portion of the length of the insert 124. The chamber wall 104 of the exhaust gas conduit 101 includes slots (not shown) which are complementary to the protrusions 125 of the chamber insert 124. The slots are configured to receive the protrusions 125 and retain the chamber insert 124 in the required position. The chamber insert may include side walls 126 which are parallel to one another and are connected by a curved wall 127 to form the U-shaped chamber insert 124. The protrusions 125 may extend orthogonally to the insert side walls 126 and from an end of the inset side walls 126 remote from the curved wall 127 which joins the two insert side walls 126. The protrusions 125 preferably extend along a portion of a length of the chamber insert 124. The side walls 126 include a stepped down portion 128 which is configured to co-operate with a wall of a turbine housing 114 to provide a flow path for exhaust gases to pass into the chamber 103. It will be appreciated that the reverse configuration in which the chamber insert 124 includes slots and the exhaust gas conduit 101 includes complementary protrusions which engage with the protrusions is also possible.

FIGS. 11a and 11b depict an embodiment of the exhaust gas conduit 101 in which the chamber 103 is defined by a sleeve 129. The sleeve 129 includes a continuous wall which encircles the main passage 105 of the exhaust gas conduit 101, save for openings required for elements such as a wastegate passage outlet or a reducing agent injection point. The wall of the sleeve 129 includes an inwardly extending portion 130 which forms the chamber wall 104 of the chamber 103. By providing a sleeve 129 which encircles the main passage and has radially inwardly extending portion 130 defining the chamber wall 104, this allows for the sleeve to be separately manufactured from the remainder of the exhaust gas conduit and can allow for the selection of different sleeves depending on specific requirements.

FIG. 12 depicts an embodiment of the exhaust gas conduit 101 in which the chamber 103 is cast or 3D printed.

FIG. 13a depicts an embodiment of an exhaust gas conduit 101 which includes a ramp 131. A wall 132 is provided which partially surrounds an opening 133 configured to receive an exhaust gas sensor. The wall 132 is contiguous with the ramp 131. The wall 132 may include a first section 132a and an opposing second section 132b that are connected via the ramp 131. The wall 132 is generally circular in cross-section such that it is conformal to the geometry of the exhaust gas sensor (not shown) which is also circular. However, in alternative embodiments, any suitable cross-sectional shape of sensor may be used, and the wall 132 may be shaped conformally in dependence upon the shape of the sensor. The ramp 131 and the wall 132 together define a volume for receiving an exhaust gas sensor. The volume is generally cylindrically shaped, for conformance with the exhaust gas sensor, and is substantially surrounded by the wall 132. The opening 133 opens into the volume formed by the ramp 131 and the wall 132. The ramp 131 and the wall 132 function to shield an exhaust gas sensor disposed within the volume defined by the ramp 131 and the wall 132 from a main flow MF of exhaust gases passing through the exhaust gas conduit. The wall 132 includes a discontinuity 134 in the form of a downstream gap. The discontinuity 134 allows exhaust gases which have been deflected over the top of an exhaust gas sensor to spill into the volume defined by the ramp 131 and the wall 132 in which the exhaust gas sensor is disposed to allow the exhaust gas sensor to measure a property of the exhaust gases. The ramp 131 and the wall 132 are sized to extend into the exhaust gas conduit by a greater amount than an exhaust gas sensor such that in an assembled condition, a tip of the exhaust gas sensor is recessed relative to the ramp 131 and the wall 132.

FIG. 13b depicts a model flow of exhaust gases passing over the ramp 131. The darker flow indicates a higher velocity and demonstrates how the highest velocity gas passes along an inner wall of the exhaust gas conduit and that this layer of high-velocity gas is deflected by the ramp 131, but a portion of the exhaust gases are able to spill towards the exhaust gas sensor 107 at low velocity, thereby providing a more favourable environment for the exhaust gas sensor. This allows the exhaust gas sensor 107 to be positioned further upstream that would otherwise be the case.

FIG. 13c depicts an embodiment of an exhaust gas conduit 101 comprising a ramp 131 shown from the outside of the exhaust gas conduit 101. The height of the ramp 131 increases in a downstream direction in order to protect the exhaust gas sensor 107 from the high velocity exhaust gases.

FIGS. 14a and 14b depict an embodiment of a shield 135 for an exhaust gas sensor 107. The shield 135 surrounds a tip of the exhaust gas sensor 107. By surrounding the tip of the exhaust gas sensor 107, the shield 135 protects the tip from the high velocity exhaust gases passing by. The shield 135 is configured to control the amount of exhaust gases reaching the tip. As shown in FIG. 14b, the shield 135 includes a plurality of protrusions 136 disposed radially around a perimeter of the shield 135. The shield 135 includes a plurality of apertures 137 in a side wall of the shield 135. Each protrusion 136 may have a corresponding aperture 137.

FIG. 15 depicts an embodiment of an exhaust gas conduit 101 attached to a turbocharger 102. The exhaust gas conduit 101 differs from the embodiment shown in FIG. 2 in that the chamber 103 comprises a plurality of inlet openings 112. The plurality of inlet openings 112 are each defined by a respective opening in the main wall 109 of the exhaust gas conduit 101. Although not shown, the inlet openings 112 may comprise scoops to control and/or increase the amount of the aliquot of exhaust gases that are received by the chamber 103. The plurality of inlet openings 112 are axially aligned relative to the centreline 139 of the exhaust gas conduit 101. Although not clearly visible in FIG. 15, the chamber 103 comprises a generally toroidal passage which extends around a perimeter of the exhaust gas conduit 101 and provides fluid communication between all of the plurality of inlet openings 112 (for clarity, only some of the plurality of inlet openings 112 have been labelled in FIG. 15). In this sense, the toroidal passage functions as a manifold. The toroidal passage provides fluid communication between the inlet openings 112 and the portion of the chamber 103 which is configured to receive a portion of the exhaust gas sensor 107.

The plurality of inlet openings 112 will create a disturbance to the main flow as it passes over the inlet openings 112. In general, increasing the size of the inlet openings 112 increases the amount of aliquot of exhaust gas that can be received, however this also increases the disturbance to the main flow. This disturbance could lead to unwanted turbulence which exerts a back pressure on the turbine 117. In the present embodiment, because the chamber 103 comprises multiple inlet openings 112 the effective inlet area from which the chamber 103 can receive the aliquot of exhaust gases from is increased whilst the size of each inlet openings 112 remains relatively small. As such, each individual inlet opening 112 presents a relatively small disturbance. In the present embodiment, the chamber 103 comprises 12 inlet openings 112. However, in alternative embodiments substantially any number of auxiliary inlet openings may be used according to requirements.

Preferably, the inlet openings 112 are generally equally spaced about the exhaust gas conduit centreline 139. Spacing the inlet openings 112 equally ensures that the disturbances to main flow caused by the inlet openings 112 are the maximum distance apart from one another, so that the overall disturbance is spread out. However, in alternative embodiments uneven spacing may be used.

FIG. 16 shows a further embodiment of a turbine 7000. The turbine 7000 comprises a turbine housing 7002, a turbine wheel (not shown), a wastegate arrangement 7004, a connection adapter 7006, a dosing module 7008, and a NOx sensor 7010.

The turbine housing 7002 defines a pair of inlet volutes 7012 and a turbine wheel chamber 7014. In other embodiments, the turbine housing 7002 may define a single inlet volute. Although the turbine wheel is not shown, it will be appreciated that during use the turbine wheel sits within the turbine wheel chamber 7014 where it is supported for rotation relative to the turbine housing 7002 by a shaft (not shown) about a turbine axis 7015. Exhaust gas received from an internal combustion engine (not shown) is delivered via the inlet volutes 7012 to the turbine wheel chamber 7014 whereupon the momentum of the exhaust gas impacts the blades of the turbine wheel to generate rotation of the turbine wheel and shaft.

The connection adapter 7006 is connected to the turbine housing 7002 such that the turbine housing 7002 and connection adapter 7006 in combination define part of a turbine outlet passage 7016. The turbine outlet passage 7016 receives exhaust gas that has passed through the turbine wheel from the turbine wheel chamber 7014. The turbine outlet passage 7016 comprises a first portion 7018 that extends axially in relation to the turbine axis 7015, and a second portion 7020 that is angled relative to the first portion 7018 along an adapter flow axis 7021. The angular difference between the first and second portions 7018, 7020 (i.e. between the turbine axis 7015 and the adapter flow axis 7021) is approximately 30°, however this may be varied to suit any particular packaging requirements. In some embodiments, the second portion 7020 of the turbine outlet passage 7016 may be completely axial relative to the turbine axis 7015 such that it does not comprise any relatively angled portions.

The first portion 7018 of the turbine outlet passage 7016 is defined by the turbine housing 7002 and the second portion 7020 of the turbine outlet passage 7016 is defined by the connection adapter 7006. The second portion 7020 of the turbine outlet passage 7016 receives exhaust gas from the first portion 7018. The first portion 7018 comprises a first diffuser section 7022 and the second portion 7020 comprises a second diffuser section 7024. The first and second diffuser sections 7022, 7024 are regions of the turbine housing 7002 and connection adapter 7006 respectively in which the flow area of the turbine outlet passage 7016 (i.e. the cross-sectional area relative to the direction of flow) increases with distance from the turbine wheel.

The wastegate arrangement 7004 comprises a wastegate passage 7026 that extends between the turbine inlet volutes 7012 and the turbine outlet passage 7016. The wastegate arrangement 7004 further comprises a pair of wastegate valves 7028 which cover respective valve openings (not shown) so as to selectively permit or prevent the flow of exhaust gas through the wastegate passage 7026. The valve openings connect separately to each of the 7012 inlet volutes. The wastegate valves 7028 are mounted to a common actuator (not shown) and are controlled in unison. However, in alternative embodiments, the valves may be controlled separately. The valve openings are generally the same size, however in alternative embodiments the valve opening may be asymmetric. Moreover, the valve openings may be operated using a single valve head rather than a pair of valves 7028. During use, when the wastegate valves 7028 are open, exhaust gas from the inlet volutes 7012 is bypassed to the turbine outlet passage 7016 without passing through the turbine wheel chamber 7014 and turbine wheel.

The wastegate passage 7026 is partially defined by the connection adapter 7006. In particular, the wastegate passage 7026 joins the connection adapter 7006 at a wastegate passage outlet 7030. The wastegate passage outlet 7030 is defined in a side wall 7035 of the connection adapter 7006 and is positioned approximately at the apex of the angular bend defined between the first and second portions 7018, 2020 of the turbine outlet passage 7016 (i.e. approximately at the point at which the adapter flow axis 7021 intersects the turbine axis 7015). The wastegate passage 7026 defines a wastegate flow axis 7032 at the wastegate passage outlet 7030. The wastegate flow axis 7032 defines the direction of flow of exhaust gas from the wastegate passage 7026 as it joins the turbine outlet passage 7020. In the present embodiment, the wastegate flow axis 7032 is angled relative to the adapter flow axis 7021 by approximately 45°. However, in alternative embodiments substantially any angle may be used.

The connection adapter 7006 comprises a mount 7034 for the dosing module 7008. The mount 7034 defines an opening 7036 within which a nozzle 7038 of the dosing module 7008 is received. The nozzle 7038 is positioned so that it is radially outwards of the side wall 7035 of the connection adapter 7006. However in other embodiments the nozzle 7038 may be substantially aligned with the side wall of the connection adapter 7006. The opening 7036 is positioned within the second diffuser section 7024. The nozzle 7038 is configured to generate a spray of aftertreatment fluid which is directed into the turbine outlet passage 7016 along a spray axis 7040. The spray axis 7040 is angled at around 7° downstream relative to a normal to the adapter axis 7021, however in other embodiments the spray axis 7040 may be angled at a different angle to the adapter axis 7021, for example normal to the adapter axis 7021. The spray of aftertreatment fluid defines a spray region 7042, the presence of which is shown schematically by dotted lines in FIGS. 16 and 18.

The mount 7034 and opening 7036 for the dosing module 7008 are positioned on substantially the opposite side of the turbine outlet passage 7016 to the wastegate passage outlet 7030. Moreover, the mount 7034 and opening 7036 for the dosing module 7008 are positioned downstream of the wastegate passage outlet 7030. The position of the wastegate passage outlet 7030 relative to the spray region 7042 and the angle of the wastegate flow axis 7032 relative to the spray region 7042 are such that, during use, when the wastegate valves 7028 are open, exhaust gas that has passed through the wastegate passage 7026 is directed into the spray region 7042 so that it fluidically exchanges momentum with the injected aftertreatment fluid.

The mount 7034 and opening 7036 for the dosing module 7008 are positioned within and/or form part of the connection adapter 7006. However, in alternative embodiments the mount 7034 and opening 7036 for the dosing module 7008 may be positioned within and/or form part of the turbine housing 7002. Because the mount 7034 and opening 7036 for the dosing module 7008 are positioned within the connection adapter 7006 or the turbine housing 7002, this means that the dosing module 7008 is positioned close to the turbine wheel. Accordingly this means that the injected aftertreatment fluid may take advantage of high exhaust gas temperatures which aid evaporation and decomposition. In this regard, the mount 7034, opening 7036 and dosing module 7008 are preferably positioned within a distance of no more than around 10 turbine wheel exducer diameters downstream of the turbine wheel (preferably no more than around 5 exducer diameters, and more preferably no more than around 3 exducer diameters). In this context, a turbine wheel exducer diameter is the diameter of the exducer portion of the turbine wheel, which is approximately equal to the diameter of the narrowest portion of the first diffuser section 7022. In the illustrated embodiment the mount 7034, opening 7036 and dosing module 7008 are positioned at a distance of around 3.3 exducer diameters downstream of the downstream end of the turbine wheel chamber (and wheel).

The connection adapter 7006 comprises a sensor conduit 7044 having a sensor conduit inlet 7046 configured to receive an aliquot of exhaust gas from the turbine outlet passage 7016 and sensor conduit outlet 7048 configured to re-introduce exhaust gas from the sensor conduit 7044 to the turbine outlet passage 7016. The sensor conduit 7044 defines a flow area that is larger than the size of the sensor conduit inlet 7046. Accordingly, the sensor conduit 7044 acts to decelerate the exhaust gas passing therethrough. The sensor conduit comprises a mount 7050 configured to receive the NOx sensor 7010. The NOx sensor 7010 comprises a sensing tip 7052 which protrudes into the interior of the sensor conduit 7044. Because the geometry of the sensing conduit 7044 decelerates the exhaust gas passing therethrough, the sensing tip 7052 is exposed to lower velocity exhaust gas, thus reducing the risk of damage to the sensing tip 7052 and improving the accuracy of sensor readings.

It will be appreciated that because the sensor conduit 7044 decelerates the flow therethrough, the turbine outlet passage 7016 of FIGS. 16 to 19 is an exemplary embodiment of an exhaust gas conduit according to the first aspect of the invention. It will be appreciated that the sensor conduit 7044 may therefore have a similar or the same construction as the chamber 103 (or exhaust gas sensor channel) of any of FIGS. 2 to 12. In particular, the sensor conduit 7044 may be considered to define a chamber configured to modify the velocity and/or the pressure of exhaust gases passing therethrough within the meaning of the first aspect of the invention. Likewise, the mount 7050 may be considered to define a mounting point in the chamber for mounting an exhaust gas sensor.

With reference to FIG. 16, the sensor conduit inlet 7046 is positioned upstream of the opening 7036 for the dosing module 7008. Accordingly, the risk of aftertreatment entering the sensor conduit 7044 and adversely affecting readings taken by the NOx sensor 7010 is eliminated. The sensor conduit 7044 is part of the second diffuser section 7024. However, in alternative embodiments the sensor conduit 7044 may be part of the first diffuser section 7022.

FIG. 20 shows a further embodiment of a turbine 8000. The turbine 8000 comprises a turbine housing 8002, a turbine wheel (not shown), a variable geometry mechanism (not shown), a connection adapter 8006, a dosing module 8008, and a NOx sensor 8010.

The turbine housing 8002 defines an inlet volute 8012 and a turbine wheel chamber 8014. In other embodiments, the turbine housing 8002 may define more than one inlet volute 8012. Although the turbine wheel is not shown, it will be appreciated that during use the turbine wheel sits within the turbine wheel chamber 8014 where it is supported for rotation relative to the turbine housing 8002 by a shaft (not shown) about a turbine axis 8015. Exhaust gas received from an internal combustion engine (not shown) is delivered via the inlet volute 8012 to the turbine wheel chamber 8014 whereupon the momentum of the exhaust gas impacts the blades of the turbine wheel to generate rotation of the turbine wheel and shaft.

The connection adapter 8006 is connected to the turbine housing 8002 such that the turbine housing 8002 and connection adapter 8006 in combination define part of a turbine outlet passage 8016. The turbine outlet passage 8016 receives exhaust gas that has passed through the turbine wheel from the turbine wheel chamber 8014. The turbine outlet passage 8016 comprises a first portion 8018 is defined by the turbine housing 8002, and a second portion 8020 that is defined by the connection adapter 8006. The second portion 8020 of the turbine outlet passage 8016 receives exhaust gas from the first portion 8018. The first portion 8018 comprises a first diffuser section 8022 and the second portion 8020 comprises a second diffuser section 8024. The first and second diffuser sections 8022, 8024 are regions of the turbine housing 8002 and connection adapter 8006 respectively in which the flow area of the turbine outlet passage 8016 (i.e. the cross-sectional area relative to the direction of flow) increases with distance from the turbine wheel. The first and second diffuser sections 8022, 8024 are substantially continuous with one another so as to define a single continuous diffuser.

With reference to FIG. 21, the connection adapter 8006 comprises a mount 8034 for the dosing module 8008. The mount 8034 defines an opening 8036 within which a nozzle 8038 of the dosing module 8008 is received. The nozzle 8038 is positioned so that it is radially outwards of the side wall 8035 of the connection adapter 8006. However in other embodiments the nozzle 8038 may be substantially aligned with the side wall of the connection adapter 8006. The opening 8036 is positioned within the second diffuser section 8024. The nozzle 8038 is configured to generate a spray of aftertreatment fluid which is directed into the turbine outlet passage 8016 along a spray axis 8040. The spray axis 8040 is angled at around 7° downstream relative to a normal to the adapter axis 8021, however in other embodiments the spray axis 8040 may be angled at a different angle to the adapter axis 8021, for example normal to the adapter axis 8021. The spray of aftertreatment fluid defines a spray region 8042, the presence of which is shown schematically by dotted lines in FIG. 21. The mount 8034 and opening 8036 for the dosing module 8008 are positioned within the connection adapter 8006. However, in alternative embodiments the mount 8034 and opening 8036 for the dosing module 8008 may be positioned within the turbine housing 8002.

Because the mount 8034 and opening 8036 for the dosing module 8008 are positioned within the connection adapter 8006 or the turbine housing 8002, this means that the dosing module 8008 is positioned close to the turbine wheel. Accordingly this means that the injected aftertreatment fluid may take advantage of high exhaust gas temperatures which aid evaporation and decomposition. In this regard, the mount 8034, opening 8036 and dosing module 8008 are preferably positioned within a distance of no more than around 10 turbine wheel exducer diameters downstream of the turbine wheel (preferably no more than around 5 exducer diameters, and more preferably no more than around 3 exducer diameters). In this context, a turbine wheel exducer diameter is the diameter of the exducer portion of the turbine wheel, which is approximately equal to the diameter of the narrowest portion of the first diffuser section 8022. In the illustrated embodiment, the mount 8034, opening 8036 and dosing module 8008 are positioned within around 1.7 exducer diameters of the downstream end of the turbine wheel and turbine wheel chamber 8014. In other embodiments, the mount 8034, opening 8036 and dosing module 8008 may be positioned anywhere up to around 2 exducer diameters of the downstream end of the turbine wheel and turbine wheel chamber 8014, and are preferably located at least 1 exducer diameter downstream of the downstream end of the turbine wheel and turbine wheel chamber 8014.

The connection adapter 8006 comprises a sensor conduit 8044 having a sensor conduit inlet 8046 configured to receive an aliquot of exhaust gas from the turbine outlet passage 8016 and sensor conduit outlet 8048 configured to re-introduce exhaust gas from the sensor conduit 8044 to the turbine outlet passage 8016. The sensor conduit inlet 8046 is angled relative to a normal of the turbine axis 8015 so as to define a generally scooped shape relative to the direction of flow. The angled profile of the sensor conduit inlet 8046 provides a greater area for fluid ingress into the sensor conduit 8044 whilst ensuring that the profile of the sensor conduit does not overly protrude into the turbine outlet passage 8016 where it may cause an impediment to flow. The sensor conduit outlet 8048 is angled generally normal to the turbine axis 8015. The sensor conduit 8044 defines a flow area that is larger than the size of the sensor conduit inlet 8046. Accordingly, the sensor conduit 8044 acts to decelerate the exhaust gas passing therethrough. The sensor conduit comprises a mount 8050 configured to receive the NOx sensor 8010. The NOx sensor 8010 comprises a sensing tip 8052 which protrudes into the interior of the sensor conduit 8044. Because the geometry of the sensing conduit 8044 decelerates the exhaust gas passing therethrough, the sensing tip 8052 is exposed to lower velocity exhaust gas, thus reducing the risk of damage to the sensing tip 8052 and improving the accuracy of sensor readings.

It will be appreciated that because the sensor conduit 8044 decelerates the flow therethrough, the turbine outlet passage 8016 of FIGS. 20 to 22 is an exemplary embodiment of an exhaust gas conduit according to the first aspect of the invention. It will be appreciated that the sensor conduit 8044 may therefore have a similar or the same construction as the chamber 103 (or exhaust gas sensor channel) of any of FIGS. 2 to 12. In particular, the sensor conduit 8044 may be considered to define a chamber configured to modify the velocity and/or the pressure of exhaust gases passing therethrough within the meaning of the first aspect of the invention. Likewise, the mount 8050 may be considered to define a mounting point in the chamber for mounting an exhaust gas sensor.

With reference to FIG. 21, the sensor conduit inlet 8046 is positioned upstream of the opening 8036 for the dosing module 8008. Accordingly, the risk of aftertreatment entering the sensor conduit 8044 and adversely affecting readings taken by the NOx sensor 8010 is eliminated. The sensor conduit 8044 is part of the second diffuser section 8024. However, in alternative embodiments the sensor conduit 8044 may be part of the first diffuser section 8022.

Although not shown in illustrated embodiments, the components which come into contact with the exhaust gases and/or the reductant, for example DEF, may be at least partly formed from, or lined with, stainless steel. This is desirable for the reason that stainless steel is resistant to corrosion from byproducts formed by the injected aftertreatment fluid/reductant. It is therefore desirable that components which are subject to the greatest exposure to the DEF be formed from stainless steel, or be lined with stainless steel.

Embodiments described in this application provide a number of advantages including: 1) the ability to provide an exhaust gas sensor further upstream than would otherwise be the case; 2) the ability to inject reducing agent into the exhaust stream without it affecting the reading of the exhaust gas sensors, particularly NOx sensors; 3) increasing the longevity of exhaust gas sensors and other sensors by preventing high velocity flows from contacting said sensors.

The described and illustrated embodiments are to be considered as illustrative and not restrictive in character, it being understood that only preferred embodiments have been shown and described and that all changes and modifications that come within the scope of the inventions as defined in the claims are desired to be protected. In relation to the claims, it is intended that when words such as “a,” “an,” “at least one,” or “at least one portion” are used to preface a feature there is no intention to limit the claim to only one such feature unless specifically stated to the contrary in the claim. When the language “at least a portion” and/or “a portion” is used the item can include a portion and/or the entire item unless specifically stated to the contrary.

Optional and/or preferred features as set out herein may be used either individually or in combination with each other where appropriate and particularly in the combinations as set out in the accompanying claims. The optional and/or preferred features for each aspect of the invention set out herein are also applicable to any other aspects of the invention, where appropriate.

1. An exhaust gas conduit for an exhaust system of an internal combustion engine, said exhaust gas conduit including a main passage for a main flow of exhaust gases passing through the exhaust gas conduit, a chamber configured to receive an aliquot of exhaust gases separated from the main flow of exhaust gases, a mounting point in the chamber for mounting an exhaust gas sensor, the chamber being configured to modify the velocity and/or the pressure of exhaust gases passing therethrough.

2. An exhaust gas conduit according to claim 1, wherein the chamber is configured to reduce the velocity and/or the pressure of the exhaust gases passing therethrough.

3. The exhaust gas conduit according to claim 1, wherein an exhaust gas sensor is at least partially disposed in the chamber.

4. The exhaust gas conduit according to claim 3, wherein the exhaust gas sensor is a NOx sensor.

5. The exhaust gas conduit according to claim 1, wherein the chamber is provided at least partially in a recess in a wall of the exhaust gas conduit.

6. The exhaust gas conduit according to claim 1, wherein the chamber is at least partially defined by a wall which separates exhaust gases within the chamber from the remainder of the exhaust gases in the exhaust gas conduit.

7. The exhaust gas conduit according to claim 1, wherein the chamber is configured to return the aliquot of exhaust gases to the main flow of exhaust gases.

8. The exhaust gas conduit according to claim 1, wherein the chamber comprises an inlet opening and an outlet opening.

9. The exhaust gas conduit according to claim 8, wherein the inlet opening is disposed upstream of a reducing agent injection point.

10. The exhaust gas conduit according to claim 9, wherein the inlet opening is separated from the reducing agent injection point by a distance sufficient such that in operation essentially no reducing agent provided via the reducing agent injection point enters the chamber.

11. The exhaust gas conduit according to claim 9, wherein the exhaust gas system comprises a turbine wheel and the turbine wheel comprises an exducer defining an exducer diameter, the exhaust gas conduit defines a centreline; and
wherein the inlet opening is spaced upstream of the reducing agent injection point by a distance between around 0.75 to around 1.25 exducer diameters along a centreline of the exhaust gas conduit.

12. The exhaust gas conduit according to claim 1, wherein the chamber is configured to receive a portion of exhaust gases passing along an interior face of the exhaust gas conduit.

13. The exhaust gas conduit according to claim 1, wherein the exhaust gas conduit is a diffuser, optionally wherein the exhaust gas conduit is a turbine diffuser.

14. The exhaust gas conduit according to claim 13, the exhaust gas conduit including an inlet for receiving exhaust gases from a turbocharger having a turbine wheel, the turbine wheel comprises an exducer defining an exducer diameter, the exhaust gas conduit defines a centreline; and
the chamber has an inlet opening spaced apart from the exducer of the turbine wheel by a distance of at most around 3 exducer diameters along the centreline of the exhaust gas conduit.

15. The exhaust gas conduit according to claim 1, wherein the exhaust gas conduit is an element of a turbine housing, optionally wherein the exhaust gas conduit is integral with the turbine housing.

16. The exhaust gas conduit according to claim 1, wherein the chamber is an expansion chamber.

17. (canceled)

18. The exhaust gas conduit according to claim 1, wherein the chamber is defined by a wall which separates the chamber from the main passage of the exhaust gas conduit, wherein the wall comprises an upstream edge comprising a lip configured to direct exhaust gases into the chamber.

19. The exhaust gas conduit according to claim 18, wherein the lip extends into the main passage of the exhaust gas conduit.

20. (canceled)

21. (canceled)

22. (canceled)

23. (canceled)

24. The exhaust gas conduit according to claim 1, wherein the exhaust gas conduit includes one or both of a chamber inlet slope and a chamber outlet slope.

25. (canceled)

26. (canceled)

27. (canceled)

28. (canceled)

29. A method of measuring a property of an exhaust gas of an internal combustion engine, the method including the steps of:
a) separating an aliquot of exhaust gases from a main flow of exhaust gases in an exhaust gas conduit into a chamber;
b) modifying the pressure and/or the velocity of the aliquot of exhaust gases;
c) measuring a property of the aliquot of exhaust gases.

30-51. (canceled)
